(19) United States
(12) Patent Application Publication
(10) Pub. No.: US 2023/0181580 A1
(43) Pub. Date: Jun. 15, 2023

(54) TREATMENT OF PROLIFERATIVE DISEASES OF THE CNS

(71) Applicant: AUCENTRA THERAPEUTICS PTY LTD, Adelaide (AU)

(72) Inventor: Shudong WANG, Adelaide (AU)

(21) Appl. No.: 17/923,666

(22) PCT Filed: May 5, 2021
(86) PCT No.: PCT/AU2021/000036

§ 371 Date: Nov. 7, 2022

(30) Foreign Application Priority Data
May 6, 2020 (AU) 2020901435

(51) Int. Cl. A61K 31/506; A61P 35/00; A61P 25/00; A61K 31/495; A61K 31/439; A61K 31/4439; A61K 31/4184

(52) U.S. Cl. CPC A61K 31/506; A61P 35/00; A61P 25/00; A61K 31/495; A61K 31/439; A61K 31/4439; A61K 31/4184

(57) ABSTRACT

A class of thiazole-pyrimidine compounds is disclosed for use in the treatment of proliferative cell diseases and conditions in the CNS including glioblastoma. The compounds are considered to be capable of blocking tumour cell proliferation by inhibiting the activity of CDK4 and/or CDK6 and are capable of crossing the blood brain barrier. The compounds have the general structure I: (I)

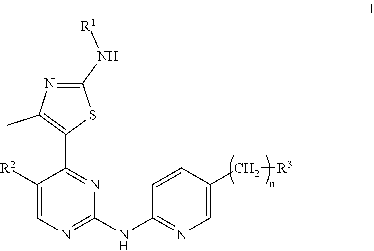

### TECHNICAL FIELD

The present disclosure relates to methods and uses of a class of thiazole-pyrimidine compounds in treating proliferative cell diseases or conditions of the central nervous system (CNS).

### PRIORITY DOCUMENT

The present application claims priority from Australian Provisional Patent Application No 2020901435 titled “Treatment of proliferative diseases of the CNS” and filed on 6 May 2020, the content of which is hereby incorporated by reference in its entirety.

### BACKGROUND

Primary brain tumours consist of a diverse group of neoplasms, derived from various cell lineages. Pursuant to a World Health Organization (WHO) categorisation, tumours of the central nervous system (CNS) are classified as astrocytic, oligodendroglial, or mixed. These tumours are further classified by subtypes and are graded, based on histology, from I to IV, with grade IV being the most aggressive. In the United States alone, it has been estimated that nearly 80,000 new cases of primary brain tumour and other CNS tumours are diagnosed every year. Sadly, these cancers are characterised by a poor prognosis and low rate of survival; glioblastoma multiforme (GBM) is the most aggressive primary tumour of the CNS, accounting for 45% of malignant primary CNS tumours and 54% of all gliomas. While survival rates for most cancers have continued to improve in recent years, cancers of the CNS are not yet experiencing the same level of success. For example, patients diagnosed with brain cancer between 2009 and 2013 had around a 25% chance of surviving for five years. This starkly contrasts with a survival rate of about 68% for all cancers combined in the same period. For GBM patients, the median survival rate is only 15-23 months and the 5-year survival is about 4.6%, which is the lowest rate of all of the brain tumour types.

Aberrant cell-cycle control, resulting in unlimited cell cycle re-entry and progression, is a hallmark of human cancers. Cyclin-dependent kinases (CDKs) are known to be associated with various cyclin subunits, playing pivotal roles in the regulation of a variety of important regulatory pathways in cells, including cell-cycle control, apoptosis, neuronal physiology, differentiation and transcription. To date, at least 20 CDKs and 30 cyclins have been identified. They may be classified into two major groups, reflecting their functions, the cell cycle regulator CDKs and the transcription regulator CDKs (Wang S et al., Trends Pharmacol Sci 29(6):302-313, 2008). The class of the cell cycle regulator CDKs includes CDK1, CDK2, CDK3, CDK4, CDK5, CDK6, and CDK7 and these function with their cyclin partners (eg cyclin A, B, C, D1, D2, D3, E, F) to regulate promotion of the cell cycle. The class of the transcription regulator CDKs includes CDK7, CDK8, CDK9 and CDK11, which work together with cyclin C, H, K, L1, L2, T1 and T2 and tend to play roles in transcriptional regulation. Given the functions of the CDK classes, it is not surprising that CDKs have been implicated in cell proliferation diseases and conditions, particularly cancer. Cell proliferation is a result of the direct or indirect deregulation of the cell division cycle and the CDKs play a critical role in the regulation of the various phases of this cycle. Therefore, inhibitors of CDKs and their associated cyclins are considered to be useful targets for cancer therapy.

CDK4/6 controls the cell cycle and is tightly regulated by the INK4 family of proteins. Numerous studies have established that the CDK4/6 pathway is hyper-activated in the vast majority of cancers, including CNS tumours and glioma (Xu G et al., J Neurooncol 136: 445-452, 2018; Parsons D W et al., Science 321:1807-1812, 2008; Bax D A et al., Clin Cancer Res 16:3368-3377, 2010; and Cancer Genome Atlas Research, N. Nature 455:1061-1068, 2008). From genome-scale profiling of glioma in children and adults, the CDK4/6-Rb axis is deregulated in >80% of GBM, which arises from/z (i) deletion of the CDKN2A/B genes that encode p16INK4a and p15INK4b, (ii) amplification/over-expression of CDK4/6, and (iii) deletion/mutation of Rb (Schmidt E E et al., Cancer Res 54:6321-6324, 1994). CDK4, p16INK4a and Rb are independent predictors of poor survival (Aoki K et al., Neuro Oncol 20:66-77, 2018). Therefore, the inhibition of CDK4/6 may be an effective approach for treating cancers particularly glioblastoma. Three CDK4/6 inhibitors, namely palbociclib, ribociclib and abemaciclib have been approved by the US Food and Drug Administration (FDA) for the treatment of breast cancer and have been trialled in GBM patients. However, the outcome with palbociclib has been disappointing and the trials terminated. The lack of efficacy may have been due to its limited ability to cross the blood-brain barrier (BBB), and thus drug exposure in the brain (Karen E et al., In 2013 AACR-NCI-EORTC International Conference on Molecular Targets and Cancer Therapeutics, Vol. 12(11 Suppl) 2013).

Cell signalling through growth factor receptors and protein kinases is another important regulator of cell growth and proliferation. In normal cell growth, growth factors, through receptor activation (ie PDGF or EGF and others), activate MAP kinase pathways. One of the most important MAP kinase pathways involved in normal and uncontrolled cell growth is the Ras/Raf kinase pathway. Active GTP-bound Res results in the activation and indirect phosphorylation of Raf kinase. Raf then phosphorylates MEK1 and MEK2 (Ahn et al., Methods Enzymol 332:417-431, 2001). The activated MEK then phosphorylates ERK 1 and ERK2. Subsequently, the phosphorylated ERK dimerises and then translocates to the nucleus where it accumulates (Khokhlatchev et al., Cell 93:605-615, 1998), and where it is then involved in several important cellular functions, including nuclear transport, signal transduction, DNA repair, nucleosome assembly and translocation, and mRNA processing and translation (Ahn et al., Molecular Cell 6:1343-1354, 2000). Overall, treatment with growth factors leads to the activation of ERK1/2 which results in proliferation and resistance to therapy. Therefore, targeting the MAP kinase pathways offers a therapeutic opportunity for a range of cancer types, and recently, a MEK inhibitor, selumetinib, has been granted US Breakthrough Therapy Designation for the treatment of patients with neurofibromatosis type 1 (NF1) symptomatic and/or progressive, inoperable plexiform neurofibromas, an incurable genetic condition. The NF1 gene mutation may result in dysregulations in RAS/RAF/MEK/ERK signalling, which can cause cells to grow, divide and copy themselves in an uncontrolled manner, and thus result in tumour growth. Selumetinib inhibits the MEK enzyme leading to inhibition of tumour growth.

In cancer cells, a major consequence of disrupted signalling pathways is an imbalance in protein expression that allows the cells to evade apoptosis, proliferate, and metastasise. Approximately 40% of GBM subtype tumours are characterised by dysfunctional EGFR, a cell surface receptor tyrosine kinase that activates a cascade of downstream intracellular signalling through the PI3K/AKT pathway. Its amplification and over-expression via mutations promotes glioma growth and survival, assisted by angiogenesis, migration and metastasis. Hence, EGFR has been proposed as an attractive therapeutic target. However, a phase II study of the EGFR inhibitor, erlotinib, in patients with recurrent GBM showed no significant benefit. Other tyrosine kinase inhibitors tested in clinical phase II-III studies, such as enzastaurin, an inhibitor targeting PKC and PI3K/AKT when used alone or in combination with chemotherapy, also demonstrated no effect in GBM patients. The failure of these EGFR inhibitor compounds is likely due to poor pharmacokinetics (PK) and BBB permeability.

CNS drugs typically have a much lower approval rate than non-CNS drugs, and attrition rates for oncology CNS drug development are very high. Thus, discovery of drugs for treating brain tumours is characterised by major obstacles and historical failure. For successful treatment, a drug must first be able to cross the BBB, a layer of closely connected endothelial cells unique to blood capillaries in the brain that are closely connected by tight junctions preventing para-cellular movement. Passage of a compound into the brain through the endothelial cells can be restricted by the action of ATP binding cassette (ABC) transporters expressed at their apical membranes; that is, at the membrane in contact with circulating blood. Of these, P-glycoprotein (P-gp) and Breast Cancer Resistance Protein (BCRP) are the two dominant transporters that, together, restrict the brain penetration of numerous compounds (Wager et al., Expert Opin Drug Discov 6:371-381, 2011; Agarwal et al., J Pharmacol Exp Ther 336:223-233, 2011). Indeed, most chemotherapeutic agents are prevented from accessing the brain by this barrier.

The location of GBM and its widespread infiltration of normal surrounding brain tissue means that surgical resection is unable to completely remove the tumours. Moreover, the tumour cells that invade the surrounding normal brain are protected by the BBB from therapeutic agents. Unfortunately, those that do have access, such as temozolomide (TMZ, the only standard-of-care chemotherapy for glioblastoma) have limited efficacy, and only improve survival by several months at best. So far, the majority of clinical trials for GBM therapies have failed. Preclinical data showed that penetration of CDK4/6 inhibitors (ie palbociclib and abemaciclib) is restricted by active efflux at the BBB. Abemaciclib showed a relatively higher exposure than palbociclib in the rodent brain, however, its therapeutic potential remains to be revealed. Clearly, there is a need to identify drugs directed towards new targets, such as CDK4/6, and which are also sufficiently able to cross the BBB to render them effective for treating brain tumours and other cancers of the CNS.

The present applicant has identified a class of thiazole-pyrimidine compounds for use in the treatment of proliferative cell diseases and conditions in the CNS including glioblastoma. While not wishing to be bound by theory, it is considered that these compounds block tumour cell proliferation by inhibiting the activity of CDK4 and/or CDK6 and are capable of crossing the BBB.

### SUMMARY

According to a first aspect, the present disclosure provides a method of treating a proliferative cell disease or condition of the central nervous system (CNS) in a subject, comprising administering to the subject a therapeutically effective amount of a compound of formula I shown below:

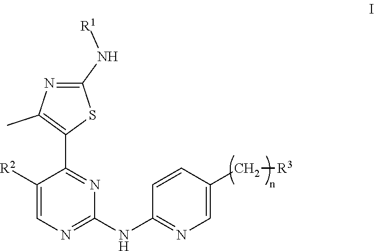

wherein:
R¹ is selected from H, alkyl, aryl, aralkyl, alicyclic, heterocyclic, halogen, NO₂, CN, CF₃, OH, O-alkyl, O-aryl, COOH, CO-alkyl, CO-aryl, CONH₂, CONH-alkyl, CONH-aryl, and CONH-alicyclic;
R² is selected from H, alkyl, halogen, NO₂, CN, CF₃, OH, O-alkyl, and NH₂;
R³ is selected from heterocyclic including at least one N heteroatom, NH-alkyl, NH-aryl, N-(alkyl)₂, N-(aryl)₂, and N-(alkyl)(aryl); and
n is an integer selected from the range of 0 to 3; and
wherein said alkyl, aryl, aralkyl, alicyclic and heterocyclic groups may be optionally substituted with one or more groups selected from alkyl, halogen, CN, OH, O-methyl, NH₂, NH-alkyl, N(alkyl)₂, COOH, COH, CO(alkyl), CONH, and CF₃;
or a pharmaceutically acceptable salt, solvate or prodrug thereof;
optionally in combination with a pharmaceutically acceptable carrier, diluent and/or excipient.

The compounds of formula I have been found to possess anti-proliferative activity (eg it is considered that these compounds block tumour cell proliferation by inhibiting the activity of CDK4 and/or CDK6) and, further, are capable of crossing the BBB.

In a second aspect, the present disclosure provides the use of a compound as defined in the first aspect, or a pharmaceutically acceptable salt, solvate or prodrug thereof, in the manufacture of a medicament for treating a proliferative cell disease or condition of the central nervous system (CNS) in a subject, for example, glioblastoma.

In a third aspect, the present disclosure provides the use of a compound as defined in the first aspect, or a pharmaceutically acceptable salt, solvate or prodrug thereof, for treating a proliferative cell disease or condition of the central nervous system (CNS) in a subject, for example, glioblastoma.

### BRIEF DESCRIPTION OF FIGURES

FIG. 1 shows anti-proliferative activity of compound 1 (5-(2-((5-(4-(dimethylamino)piperidin-1-yl)pyridin-2-yl)amino)-5-fluoropyrimidin-4-yl)-N,4-dimethylthiazol-2-amine) as a single agent and in combination with inhibitor of (A) mTOR (everolimus; denoted in the figure as “Eve”), (B) PI3K (alpelisib; “Alp”) or (C) MEK (selumetinib; “Sel”) in the T98G GBM cell line;

FIG. 2 provides the results of annexin V/PI assays of GBM U87 cells 48 hours after treatment with 5 μM palbociclib (“Palb”) or compound 1 alone or in combination with TMZ;

FIG. 3 shows the inhibition of GBM U87 cell colony formation by compound 1 through targeting of CDK4/6-mediated Rb phosphorylation;

FIG. 4 provides graphical results showing the brain uptake of compound 1 and compound 2 (N-cyclopentyl-5-(2-((5-((4-ethylpiperazin-1-yl)methyl)pyridin-2-yl)amino)-5-fiuoropyrimidin-4-yl)-4-methylthiazol-2-amine) in Balb/C mice after a 2 mg/kg intravenous dose (A), and (B) 24 hours after a 10 mg/kg oral dose; while (C) shows the brain uptake of compound 6 (N-cyclopentyl-5-(2-((5-((4-ethylpiperazin-1-yl)methyl)pyridin-2-yl)amino)pyrimidin-4-yl)-4-methylthiazol-2-amine) in Balb/C mice after a 2 mg/kg intravenous dose;

FIG. 5 provides graphical results demonstrating the in vivo anti-tumour activity of compound 1 and compound 2 on subcutaneous GBM U87 cell xenografts; and

FIG. 6 provides graphical results demonstrating the in vivo anti-tumour activity of compound 1 and compound 2 on orthotopic xenografts of GBM U87 and G4T GBM patient-derived models, respectively.

### DETAILED DESCRIPTION

According to a first aspect, the present disclosure provides a method of treating a proliferative cell disease or condition of the central nervous system (CNS) in a subject, comprising administering to the subject a therapeutically effective amount of a compound of formula I shown below:

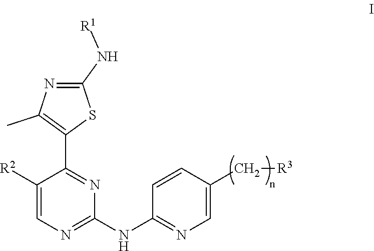

wherein:
R¹ is selected from H, alkyl, aryl, aralkyl, alicyclic, heterocyclic, halogen, NO₂, CN, CF₃, OH, O-alkyl, O-aryl, COOH, CO-alkyl, CO-aryl, CONH₂, CONH-alkyl, CONH-aryl, and CONH-alicyclic;
R² is selected from H, alkyl, halogen, NO₂, CN, CF₃, OH, O-alkyl, and NH₂;
R³ is selected from heterocyclic including at least one N heteroatom, NH-alkyl, NH-aryl, N-(alkyl)₂, N-(aryl)₂, and N-(alkyl)(aryl); and
n is an integer selected from the range of 0 to 3; and
wherein said alkyl, aryl, aralkyl, alicyclic and heterocyclic groups may be optionally substituted with one or more groups selected from alkyl, halogen, CN, OH, O-methyl, NH₂, NH-alkyl, N(alkyl)₂, COOH, COH, CO(alkyl), CONH₂ and CF₃;
or a pharmaceutically acceptable salt, solvate or prodrug thereof;
optionally in combination with a pharmaceutically acceptable carrier, diluent and/or excipient.

The compounds of formula I have been found to possess anti-proliferative activity (eg it is considered that these compounds block tumour cell proliferation by inhibiting the activity of CDK4 and/or CDK6) and, further, are capable of crossing the BBB. It is therefore considered that the compounds of formula I are useful in the treatment of proliferative cell diseases and conditions of the CNS such as glioblastoma and other diseases and conditions of the CNS associated with uncontrolled cell proliferation (or, in other words, requires control of the cell cycle). As used herein, an anti-proliferative effect within the scope of the present disclosure may be demonstrated by the ability to inhibit cell proliferation in an in vitro whole cell assay. An example(s) of such an assay, including methods for performance, are described in more detail in the examples provided hereinafter.

Preferably, the compounds of formula I modulate (eg inhibit) the activity of one or more protein kinases selected from CDK4 and/or CDK6. As mentioned above, CDK4 and CDK6 through their roles as cell cycle regulators promote cancer cell proliferation. As such, the compounds of formula I, and pharmaceutically acceptable salts, solvates and prodrugs thereof, which inhibit at least CDK4 and/or CDK6, have utility in both in vitro and in vivo applications (eg in vitro cell-based assays) and as the basis of a therapeutic method of treating cancer or another proliferative disorder or condition in a subject.

The compounds of formula I may inhibit any of the steps or stages in the cell cycle, for example, formation of the nuclear envelope, exit from the quiescent phase of the cell cycle (G0), G1 progression, chromosome decondensation, nuclear envelope breakdown, START, initiation of DNA replication, progression of DNA replication, termination of DNA replication, centrosome duplication, G2 progression, activation of mitotic or meiotic functions, chromosome condensation, centrosome separation, microtubule nucleation, spindle formation and function, interactions with microtubule motor proteins, chromatid separation and segregation, inactivation of mitotic functions, formation of contractile ring, and cytokinesis functions. In particular, the compounds of formula I may influence certain gene functions such as chromatin binding, formation of replication complexes, replication licensing, phosphorylation or other secondary modification activity, proteolytic degradation, microtubule binding, actin binding, septin binding, microtubule organising centre nucleation activity and binding to components of cell cycle signalling pathways.

In a second aspect, the present disclosure provides the use of a compound as defined in the first aspect, or a pharmaceutically acceptable salt, solvate or prodrug thereof, in the manufacture of a medicament for treating a proliferative cell disease or condition of the central nervous system (CNS) in a subject, for example, glioblastoma.

In a third aspect, the present disclosure provides the use of a compound as defined in the first aspect, or a pharmaceutically acceptable salt, solvate or prodrug thereof, for treating a proliferative cell disease or condition of the central nervous system (CNS) in a subject, for example, glioblastoma.

In this specification, a number of terms are used which are well known to those skilled in the art. Nevertheless, for the purposes of clarity, a number of these terms are hereinafter defined.

As used herein, the term “treating” includes prophylaxis as well as the alleviation of established symptoms of a disease or condition. As such, the act of “treating” a disease or condition therefore includes: (1) preventing or delaying the appearance of clinical symptoms of the disease or condition developing in a subject afflicted with or predisposed to the disease or condition; (2) inhibiting the disease or condition (ie arresting, reducing or delaying the development of the disease or condition or a relapse thereof (in case of a maintenance treatment)) or at least one clinical or subclinical symptom thereof; and (3) relieving or attenuating the disease or condition (ie causing regression of the disease or condition or at least one of its clinical or subclinical symptoms).

As used herein, the term “alkyl” includes straight chain alkyl groups, branched alkyl groups and cyclic alkyl groups having from 1 to 8 carbon atoms (eg methyl, ethyl propyl, isopropyl, butyl, isobutyl, tert-butyl, pentyl, hexyl, cyclopropyl, cyclobutyl, cyclopentyl etc).

As used herein, the term “aryl” refers to a substituted (mono- or poly-) or unsubstituted monoaromatic or polyaromatic group, wherein said polyaromatic group may be fused or unfused. The term therefore includes groups having from 6 to 10 carbon atoms (eg phenyl, naphthyl etc). It is also to be understood that the term “aryl” is synonymous with the term “aromatic”.

As used herein, the term “aralkyl” is used as a conjunction of the terms alkyl and aryl as defined above.

The term “aliphatic” takes its normal meaning in the art and includes non-aromatic groups such as alkanes, alkenes and alkynes and substituted derivatives thereof.

As used herein, the term “alicyclic” refers to a cyclic aliphatic group.

The term “halogen” refers to fluoro, chloro, bromo and iodo.

As used herein, the term “heterocyclic” refers to a saturated or unsaturated cyclic group comprising one or more heteroatoms (eg N) in the ring.

The term “derivative” as used herein, includes any chemical modification of an entity. Illustrative of such chemical modifications is the replacement of hydrogen by a halogen group, an alkyl group, an acyl group or an amino group.

As used herein, the phrase “manufacture of a medicament” includes the use of one or more of the compounds of formula I directly as the medicament or in any stage of the manufacture of a medicament comprising one or more of the compounds of formula I.

Some of the compounds of formula I may exist as single stereoisomers, racemates, and/or mixtures of enantiomers and/or diastereomers. All such single stereoisomers, racemates and mixtures thereof, are encompassed within the scope of the present disclosure. The isomeric forms such as diastereomers, enantiomers, and geometrical isomers can be separated by physical and/or chemical methods known to those skilled in the art.

The term “pharmaceutically acceptable salt” as used herein, refers to salts that retain the desired biological activity of the compounds of formula I, and include pharmaceutically acceptable acid addition salts and base addition salts. Suitable pharmaceutically acceptable acid addition salts of the compounds of formula I may be prepared from an inorganic acid or from an organic acid. Examples of such inorganic acids are hydrochloric, sulfuric and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, fumaric, maleic, alkyl sulfonic and arylsulfonic. Additional information on pharmaceutically acceptable salts can be found in Remington's Pharmaceutical Sciences, 19th Edition, Mack Publishing Co, Easton Pa. 1995.

The term “solvate” refers to any form of the compound of formula I, resulting from solvation of with an appropriate solvent. Such a form may be, for example, a crystalline solvate or a complex that may be formed between the solvent and the dissolved compound.

The term “prodrug” means a compound that undergoes conversion to a compound of formula I within a biological system, usually by metabolic means (eg by hydrolysis, reduction or oxidation). For example, an ester prodrug of a compound of formula I containing a hydroxyl group may be convertible by hydrolysis in vivo to the compound of formula I. Suitable esters of the compounds of formula I containing a hydroxyl group may be, for example, acetates, citrates, lactates, tartrates, malonates, oxalates, salicylates, propionates, succinates, fumarates, maleates, methylene-bis-P-hydroxynaphthoates, gestisates, isethionates, di-p-toluoyltartrates, methanesulfonates, ethanesulfonates, benzenesulfonates, p-toluenesulfonates, cyclohexylsulfamates and quinates. As another example, an ester prodrug of a compound of formula I containing a carboxy group may be convertible by hydrolysis in vivo to the compound of formula I. Examples of ester prodrugs include those described by Leinweber F J, Drug Melab Rev 18:379-439 (1987). Similarly, an acyl prodrug of a compound of formula I containing an amino group may be convertible by hydrolysis in vivo to the compound of formula I. Examples of prodrugs for these and other functional groups, including amines, are provided in Prodrugs: challenges and rewards, Valentino J Stella (ed), Springer, 2007.

In the case of compounds of formula I that are solid, it will be understood by those skilled in the art that the compounds (or pharmaceutically acceptable salts, solvates or prodrugs thereof) may exist in different crystalline or polymorphic forms, all of which are encompassed within the scope of the present disclosure.

The term “therapeutically effective amount” or “effective amount” is an amount sufficient to effect beneficial or desired clinical results. A therapeutically effective amount can be administered in one or more administrations. Typically, a therapeutically effective amount is sufficient for treating a disease or condition or otherwise to palliate, ameliorate, stabilise, reverse, slow or delay the progression of a disease or condition such as, for example, cancer or another proliferative cell disease or condition. By way of example only, a therapeutically effective amount of a compound of formula I, or a pharmaceutically acceptable salt, solvate or prodrug thereof, may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that the therapeutically effective amount may vary and depend upon a variety of factors including the activity of the particular compound (or salt, solvate or prodrug thereof), the metabolic stability and length of action of the particular compound (or salt, solvate or prodrug thereof), the age, body weight, sex, health, route and time of administration, rate of excretion of the particular compound (or salt, solvate or prodrug thereof), and the severity of, for example, the cancer or other proliferative cell disease or condition to be treated.

In some embodiments, R¹ is H, alkyl (eg a C₁₋₆ alkyl such as a C₁₋₃ alkyl such as methyl, ethyl and C(CH₃)₂ or a C₃₋₆ cycloalkyl such as cyclopentyl), or heterocyclic (eg a saturated or unsaturated 5- or 6-membered cyclic group comprising one or two N, O or S heteroatoms). Most preferably, R¹ is H, C₁₋₃ alkyl (eg methyl) or C₃₋₆ cycloalkyl such as cyclopentyl.

In some embodiments, R² is H, alkyl (eg a C₁₋₆ alkyl or, preferably, a C₁₋₃ alkyl such as methyl or ethyl), CN or halogen (preferably F).

In some embodiments, R³ is a heterocyclic group (preferably a saturated or unsaturated 5- or 6-membered cyclic group comprising one, but more preferably two, N heteroatoms), optionally substituted with one or more groups selected from alkyl (eg a C₁₋₆ alkyl or, preferably, a C₁₋₃ alkyl such as methyl, ethyl and CH(CH₃)₂), NH₂, NH-alkyl such as NH-methyl and NH-ethyl, N(alkyl)₂ such as N(C₁₋₃ alkyl)₂ (eg N(CH₃)₂, N(CH₂CH₃)₂ and N(CH)(CH₂CH₃)), COH and CO(C₁₋₃ alkyl) (eg COCH₃).

In some particular embodiments, R³ is selected from the following:

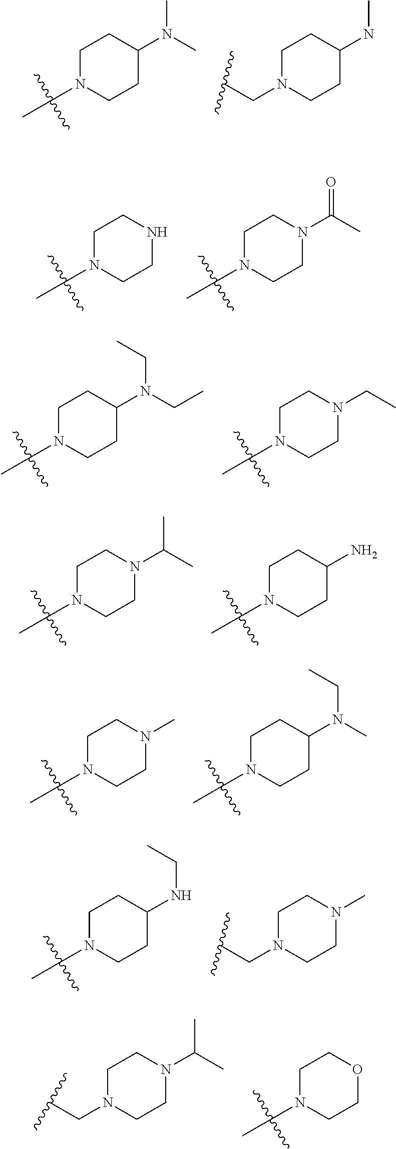

In some embodiments, n is 0, 1 or 2. Where n is 1 or 2, the compound is thereby provided with an alkyl bridge (eg a —CH₂— or —CH₂CH₂— bridge) to the carbon atom at position 4 of the pyridine ring.

In one especially preferred embodiment, the compound is:
5-(2-((5-(4-(dimethylamino)piperidin-1-yl)pyridin-2-yl)amino)-5-fluoropyrimidin-4-yl)-N,4-dimethylthiazol-2-amine;
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile;
N-cyclopentyl-5-(2-((5-((4-ethylpiperazin-1-yl)methyl)pyridin-2-yl)amino)pyrimidin-4-yl)-4-methylthiazol-2-amine;
5-(2-((5-(4-aminopiperidin-1-yl)pyridin-2-yl)amino)-5-fluoropyrimidin-4-yl)-N,4-dimethylthiazol-2-amine;
5-(2-((5-(4-aminopiperidin-1-yl)pyridin-2-yl)amino)pyrimidin-4-yl)-N-cyclopentyl-4-methylthiazol-2-amine;
N-cyclopentyl-5-(5-fluoro-2-((5-morpholinopyridin-2-yl)amino)pyrimidin-4-yl)-4-methylthiazol-2-amine;
5-(2-((5-(4-(ethylamino)piperidin-1-yl)pyridin-2-yl)amino)-5-fluoropyrimidin-4-yl)-N,4-dimethylthiazol-2-amine;
5-(2-((5-(4-(ethyl(methyl)amino)piperidin-1-yl)pyridin-2-yl)amino)-5-fluoropyrimidin-4-yl)-N,4-dimethylthiazol-2-amine;
5-(5-fluoro-2-((5-((4-methylpiperazin-1-yl)methyl)pyridin-2-yl)amino)pyrimidin-4-yl)-N,4-dimethylthiazol-2-amine;
5-(5-fluoro-2-((5-((4-isopropylpiperazin-1-yl)methyl)pyridin-2-yl)amino)pyrimidin-4-yl)-N,4-dimethylthiazol-2-amine; or
5-(2-((5-(4-(diethylamino)piperidin-1-yl)pyridin-2-yl)amino)-5-fluoropyrimidin-4-yl)-N,4-dimethylthiazol-2-amine.

In some preferred embodiments, the compounds of formula I exhibit anti-proliferative activity in human cell lines, as measured by a standard cytotoxicity assay. Preferably, the compound exhibits an IC₅₀ value of less than 5 μM, even more preferably less than 1 μM as measured by a standard cell viability assay. More preferably still, the compound exhibits an IC₅₀ value of less than 0.5 μM.

In some preferred embodiments, the compounds of formula I inhibit one or more protein kinases, as measured by any standard assay well known to those skilled in the art. Preferably, the compound exhibits an IC₅₀ value of less than 1 μM or less than 0.5 μM as measured by the kinase assay described in Example 2 hereinafter, more preferably still less than 0.1 μM.

Particular examples of compounds of formula I for use in the method of the first aspect are shown in Table 1 below.

TABLE 1
Chemical structure of selected compounds of the present disclosure

| No. | Structure | Name | Mass |
|---|---|---|---|
| 1. | 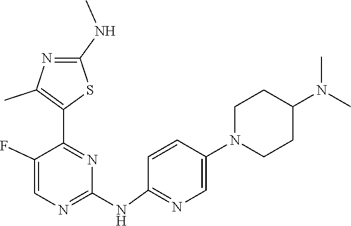 [SMILES: CNc1nc(C)c(-c2nc(Nc3ccc(N4CCC(N(C)C)CC4)cn3)ncc2F)s1] | 5-(2-((5-(4-(dimethylamino)piperidin-1-yl)pyridin-2- yl)amino)-5-fluoropyrimidin-4-yl)-N,4- dimethylthiazol-2-amine | 442.6 |
| 2. | 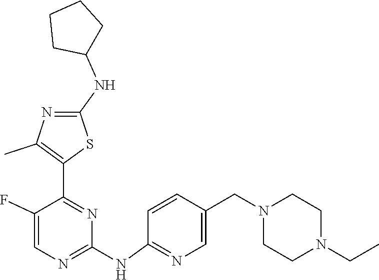 [SMILES: CCN1CCN(Cc2ccc(Nc3ncc(F)c(-c4sc(NC5CCCC5)nc4C)n3)nc2)CC1] | N-cyclopentyl-5-(2-((5-((4-ethylpiperazin-1- yl)methyl)pyridin-2-yl)amino)-5-fluoropyrimidin-4- yl)-4-methylthiazol-2-amine | 496.7 |
| 3. | 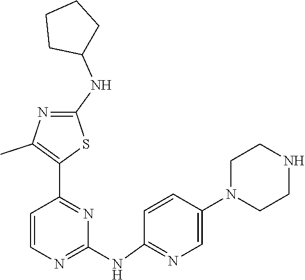 [SMILES: Cc1nc(NC2CCCC2)sc1-c1ccnc(Nc2ccc(N3CCNCC3)cn2)n1] | N-cyclopentyl-4-methyl-5-(2-((5-(piperazin-1- yl)pyridin-2-yl)amino)pyrimidin-4-yl)thiazol-2- amine | 436.2 |
| 4. | 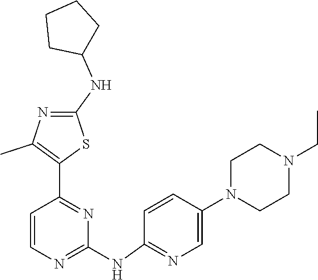 [SMILES: CCN1CCN(c2ccc(Nc3nccc(-c4sc(NC5CCCC5)nc4C)n3)nc2)CC1] | N-cyclopentyl-5-(2-((5-(4-ethylpiperazin-1- yl)pyridin-2-yl)amino)pyrimidin-4-yl)-4- methylthiazol-2-amine | 464.2 |
| 5. | 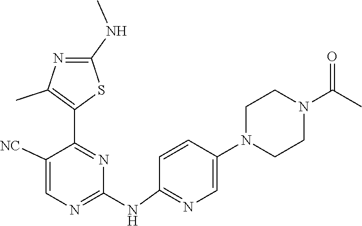 [SMILES: CNc1nc(C)c(-c2nc(Nc3ccc(N4CCN(C(C)=O)CC4)cn3)ncc2C#N)s1] | 2-((5-(4-acetylpiperazin-1-yl)pyridin-2-yl)amino)-4- (4-methyl-2-(methylamino)thiazol-5-yl)pyrimidine- 5-carbonitrile | 449.2 |
| 6. | 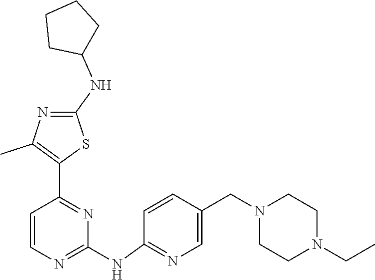 [SMILES: CCN1CCN(Cc2ccc(Nc3nccc(-c4sc(NC5CCCC5)nc4C)n3)nc2)CC1] | N-cyclopentyl-5-(2-((5-((4-ethylpiperazin-1- yl)methyl)pyridin-2-yl)amino)pyrimidin-4-yl)-4- methylthiazol-2-amine | 478.3 |
| 7. | 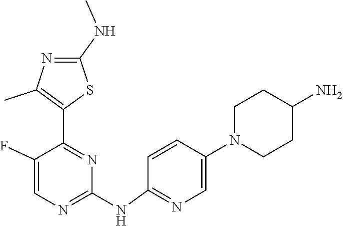 [SMILES: CNc1nc(C)c(-c2nc(Nc3ccc(N4CCC(N)CC4)cn3)ncc2F)s1] | 5-(2-((5-(4-aminopiperidin-1-yl)pyridin-2-yl)amino)- 5-fluoropyrimidin-4-yl)-N,4-dimethylthiazol-2-amine | 414.2 |
| 8. | 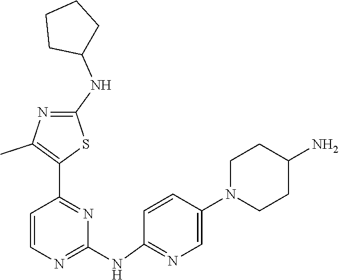 [SMILES: Cc1nc(NC2CCCC2)sc1-c1ccnc(Nc2ccc(N3CCC(N)CC3)cn2)n1] | 5-(2-((5-(4-aminopiperidin-1-yl)pyridin-2- yl)amino)pyrimidin-4-yl)-N-cyclopentyl-4- methylthiazol-2-amine | 450.2 |
| 9. | 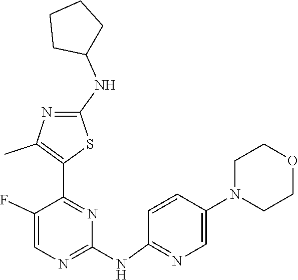 [SMILES: Cc1nc(NC2CCCC2)sc1-c1nc(Nc2ccc(N3CCOCC3)cn2)ncc1F] | N-cyclopentyl-5-(5-fluoro-2-((5-morpholinopyridin- 2-yl)amino)pyrimidin-4-yl)-4-methylthiazol-2-amine | 455.2 |
| 10. | 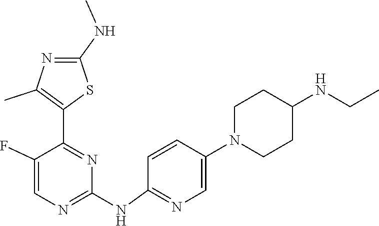 [SMILES: CCNC1CCN(c2ccc(Nc3ncc(F)c(-c4sc(NC)nc4C)n3)nc2)CC1] | 5-(2-((5-(4-(ethylamino)piperidin-1-yl)pyridin-2- yl)amino)-5-fluoropyrimidin-4-yl)-N,4- dimethylthiazol-2-amine | 442.2 |
| 11. | 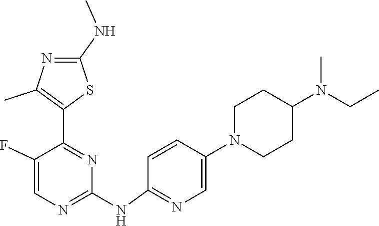 [SMILES: CCN(C)C1CCN(c2ccc(Nc3ncc(F)c(-c4sc(NC)nc4C)n3)nc2)CC1] | 5-(2-((5-(4-(ethyl(methyl)amino)piperidin-1- yl)pyridin-2-yl)amino)-5-fluoropyrimidin-4-yl)-N,4- dimethylthiazol-2-amine | 456.2 |
| 12. | 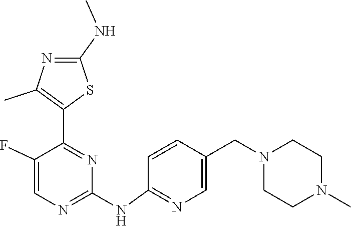 [SMILES: CNc1nc(C)c(-c2nc(Nc3ccc(CN4CCN(C)CC4)cn3)ncc2F)s1] | 5-(5-fluoro-2-((5-((4-methylpiperazin-1- yl)methyl)pyridin-2-yl)amino)pyrimidin-4-yl)-N,4- dimethylthiazol-2-amine | 428.2 |
| 13. | 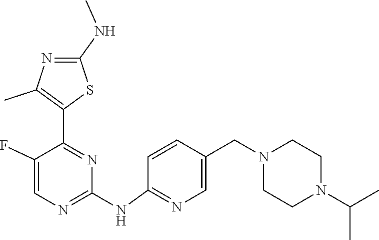 [SMILES: CNc1nc(C)c(-c2nc(Nc3ccc(CN4CCN(C(C)C)CC4)cn3)ncc2F)s1] | 5-(5-fluoro-2-((5-((4-isopropylpiperazin-1- yl)methyl)pyridin-2-yl)amino)pyrimidin-4-yl)-N,4- dimethylthiazol-2-amine | 456.2 |
| 14. | 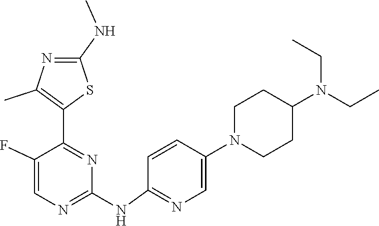 [SMILES: CCN(CC)C1CCN(c2ccc(Nc3ncc(F)c(-c4sc(NC)nc4C)n3)nc2)CC1] | 5-(2-((5-(4-(diethylamino)piperidin-1-yl)pyridin-2- yl)amino)-5-fluoropyrimidin-4-yl)-N,4- dimethylthiazol-2-amine | 470.2 |

The compounds of formula I (and pharmaceutically acceptable salts, solvates and prodrugs thereof) may be administered in combination with one or more additional agent(s) for the treatment of cancer or another proliferative disease or condition. For example, the compounds may be used in combination with other anti-cancer agents in order to inhibit more than one cancer signalling pathway simultaneously so as to make cancer cells more susceptible to anti-cancer therapies (eg treatments with other anti-cancer agents, chemotherapy, radiotherapy or a combination thereof). As such, the compounds of formula I may be used in combination with one or more of the following categories of anti-cancer agents, particularly where such anti-cancer agents are capable of crossing the BBB:
other anti-proliferative/antineoplastic drugs and combinations thereof, as used in medical oncology, such as alkylating agents (eg carmustine, procarbazine, lomustine, vincristine, and TMZ); antimetabolites (eg gemcitabine and antifolates such as fluoropyrimidines like 5-fluorouracil and tegafur, raltitrexed, methotrexate, cytosine arabinoside, fludarabine and hydroxyurea); antitumour antibiotics (eg anthracyclines such as adriamycin, bleomycin, doxorubicin, daunomycin, epirubicin, idarubicin, mitomycin-C, dactinomycin and mithramycin); antimitotic agents (eg vinca alkaloids such as vincristine, vinblastine, vindesine and vinorelbine and taxoids including taxol and taxotere and polokinase inhibitors); and topoisomerase inhibitors (eg epipodophyllotoxins such as etoposide and teniposide, amsacrine, topotecan and camptothecin);
cytostatic agents such as antioestrogens (eg tamoxifen, fulvestrant, toremifene, raloxifene, droloxifene and iodoxyfene), antiandrogens (eg bicalutamide, flutamide, nilutamide and cyproterone acetate), LHRH antagonists or LHRH agonists (eg goserelin, leuprorelin and buserelin), progestogens (eg megestrol acetate), aromatase inhibitors (eg as anastrozole, letrozole, vorazole and exemestane) and inhibitors of 5α-reductase such as finasteride;
anti-invasion agents (eg c-Src kinase family inhibitors such as 4-(6-chloro-2,3-methylenedioxyanilino)-7-[2-(4-methylpiperazin-1-yl)ethoxy]-5-tetrahydropyran-4-yloxyquinazoline (AZD0530; International Patent Publication No WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib) and bosutinib (SKI-606)), and metalloproteinase inhibitors including marimastat, inhibitors of urokinase plasminogen activator receptor function or antibodies to heparanase;
inhibitors of growth factor function (eg growth factor antibodies and growth factor receptor antibodies such as the anti-erbB2 antibody trastuzumab (Herceptin™), the anti-EGFR antibody panitumumab, the anti-erbB1 antibody cetuximab (Erbitux, C225) and any growth factor or growth factor receptor antibodies disclosed by Stern et al. Critical reviews in oncology/haematology, 2005, Vol. 54, pp 11-29). Such inhibitors also include tyrosine kinase inhibitors such as inhibitors of the epidermal growth factor family (eg EGFR family tyrosine kinase inhibitors such as N-(3-chloro-4-fluorophenyl)-7-methoxy-6-(3-morpholinopropoxy) quinazolin-4-amine (gefitinib, ZD1839), N-(3-ethynylphenyl)-6,7-bis(2-methoxyethoxy) quinazolin-4-amine (erlotinib, OSI-774) and 6-acrylamido-N-(3-chloro-4-fluorophenyl)-7-(3-morpholinopropoxy)-quinazolin-4-amine (CI 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (eg Ras/Raf signalling inhibitors such as farnesyl transferase inhibitors including sorafenib (BAY 43-9006), tipifarnib (RI 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor (insulin-like growth factor) kinase inhibitors; aurora kinase inhibitors (eg AZD1152, PH739358, VX-680, MLN8054, R763, MP235, MP529, VX-528 and AX39459) and cyclin dependent kinase inhibitors such as CDK4 and/or CDK6 inhibitors (eg palbociclib, ribociclib and abemaciclib);
antiangiogenic agents such as those which inhibit the effects of vascular endothelial growth factor (eg the anti-vascular endothelial cell growth factor antibody bevacizumab (Avastin™) and VEGF receptor tyrosine kinase inhibitors such as vandetanib (ZD6474), vatalanib (PTK787), sunitinib (SU11248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within International Patent Publication No WO 00/47212), compounds such as those disclosed in International Patent Publication Nos WO97/22596, WO 97/30035, WO 97/32856 and WO 98/13354, and compounds that work by other mechanisms (eg linomide, inhibitors of integrin αvβ3 function and angiostatin);
vascular damaging agents such as Combretastatin A4 and compounds disclosed in International Patent Publication Nos WO 99/02166, WO 00/40529, WO 00/41669, WO 01/92224, WO 02/04434 and WO 02/08213;
an endothelin receptor antagonist such as zibotentan (ZD4054) or atrasentan;
antisense therapies such as those which are directed to the targets listed above, such as ISIS 2503, an anti-ras antisense;
gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
immunotherapy approaches, including for example ex vivo and in vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

In some embodiments, the compounds of formula I (and pharmaceutically acceptable salts, solvates and prodrugs thereof) may be administered in combination with TMZ (and/or used with radiation). Using glioblastoma xenografts, it has been demonstrated that the use of abemaciclib or palbociclib (both CDK4/6 inhibitors) with TMZ or radiation inhibits DNA double-strand break repair and increases apoptosis (Raub T J et al. Drug Metab Dispos 43:1360-1371, 2015; and Michaud K et al. Cancer Res 8:70, 2010). Palbociclib is synergistic against the glioblastoma xenografts when combined with the mTOR inhibitor, everolimus (Ohmez I et al. Clinic Cancer Res DOI: 10.1158/1078-0432.CCR-17-0803, 2018). Palbociclib has also been shown to increase tumour cell antigen and antitumour T-cell responses, suggesting that there is an immunity effect of CDK4 and/or CDK6 inhibition (Deng J et al. Cancer Discovery DOI: 10.1158/2159-8290.CD-17-0915, 2018). In other embodiments, the compounds of formula I (and pharmaceutically acceptable salts, solvates and prodrugs thereof) may be administered in combination with a kinase inhibitor selected from inhibitors of PI3K, mTOR and/or MEK.

Where used in combination with other anti-cancer agents, a compound of formula I and the other anti-cancer agent can be administered in the same pharmaceutical composition or in separate pharmaceutical compositions. If administered in separate pharmaceutical compositions, the compound and the other anti-cancer agent may be administered simultaneously or sequentially in any order (eg within seconds or minutes or even hours (eg 2 to 48 hours)).

The compound of the formula I is typically applied to the treatment of cancer or another proliferative cell disease or condition in a human subject. However, the subject may also be selected from, for example, livestock animals (eg cows, horses, pigs, sheep and goats), companion animals (eg dogs and cats) and exotic animals (eg non-human primates, tigers, elephants etc).

Cancers and other proliferative cell diseases and conditions of the CNS that may be treated in accordance with the present disclosure include brain and spinal cord cancers, including glioblastomas (eg GBM), medulloblastomas, primary central nervous system (CNS) lymphoma, other malignant CNS tumours such as astrocytomas, ependymonas, oligodendrogliomas or metastatic brain tumour), and benign neoplasms of the CNS such as Schwannomas, pituitary adenomas, meningiomas and craniopharyngiomas.

In some preferred embodiments, the compounds of formula I are used to treat cancers and other proliferative cell diseases and conditions of the CNS selected from those characterised by over-expression of CDK4 and/or cyclin D including, for example, GBM. CDK4 and/or cyclin D over-expression may be determined by, for example, assessing the amount of mRNA encoding CDK4 and/or cyclin D in a suitable sample using any of the techniques well known to those skilled in the art (eg quantitative amplification techniques such as qPCR).

In some preferred embodiments, the compounds of formula I are used to treat cancers and other proliferative cell diseases and conditions of the CNS selected from those characterised by over-expression of CDK6 and/or cyclin D including, for example, medullablastomas (reviewed in Tadesse et al., Targeting CDK6 in Cancer: State of the Art and New Insights inpress). CDK6 and/or cyclin D over-expression may be determined by, for example, assessing the amount of mRNA encoding CDK6 and/or cyclin D in a suitable sample using any of the techniques well known to those skilled in the art (eg quantitative amplification techniques such as qPCR).

The compounds of formula I may be formulated into a pharmaceutical composition with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, Pa. 1995. Examples of suitable excipients for the various different forms of pharmaceutical compositions described herein may be found in the Handbook of Pharmaceutical Excipients, 2nd Edition, (1994), Edited by A Wade and P J Weller. Examples of suitable carriers include lactose, starch, glucose, methyl cellulose, magnesium stearate, mannitol, sorbitol and the like. Examples of suitable diluents include ethanol, glycerol and water. The choice of carrier, diluent and/or excipient may be made with regard to the intended route of administration and standard pharmaceutical practice.

A pharmaceutical composition comprising a compound of formula I may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, dyes and even flavouring agents may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising a compound of formula I may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration. For oral administration, particular use may be made of compressed tablets, pills, tablets, gellules, drops, and capsules. For other forms of administration, a pharmaceutical composition may comprise solutions or emulsions which may be injected intravenously, intraarterially, intrathecally, subcutaneously, intradermally, intraperitoneally or intramuscularly, and which are prepared from sterile or sterilisable solutions. A pharmaceutical composition comprising a compound of formula I may also be in form of suppositories, pessaries, suspensions, emulsions, lotions, ointments, creams, gels, sprays, solutions or dusting powders. A pharmaceutical composition may be formulated in unit dosage form (ie in the form of discrete portions containing a unit dose, or a multiple or sub-unit of a unit dose).

The compounds of formula I may be provided as a pharmaceutically acceptable salt including, for example, suitable acid addition or base salts thereof. A review of suitable pharmaceutical salts may be found in Berge et al., J Pharm Sci 66:1-19 (1977). Salts are formed, for example with strong inorganic acids such as mineral acids (eg sulfuric acid, phosphoric acid or hydrohalic acids), with strong organic carboxylic acids, such as alkanecarboxylic acids of 1 to 4 carbon atoms which are unsubstituted or substituted (eg by halogen), such as acetic acid, with saturated or unsaturated dicarboxylic acids (eg oxalic, malonic, succinic, maleic, fumaric, phthalic or tetraphthalic acid), with hydroxycarboxylic acids (eg ascorbic, glycolic, lactic, malic, tartaric or citric acid), with amino acids (eg aspartic or glutamic acid), with benzoic acid, or with organic sulfonic acids (eg (C₁-C₄)-alkyl- or aryl-sulfonic acids which are unsubstituted or substituted by, for example, halogen) such as methane- or p-toluene sulfonic acid).

The compounds of formula I may be provided in their various crystalline forms, polymorphic forms and (an)hydrous forms. In this regard, it is well known to those skilled in the art that chemical compounds may be isolated in any of such forms by slightly varying the method of purification and or isolation from the solvents used in the synthetic preparation of such compounds.

Methods for synthesising compounds of formula I have been previously described (see, for example, WO 2017/020065). In some embodiments, compounds of formula I may be synthesised by adapting the following general synthetic scheme:

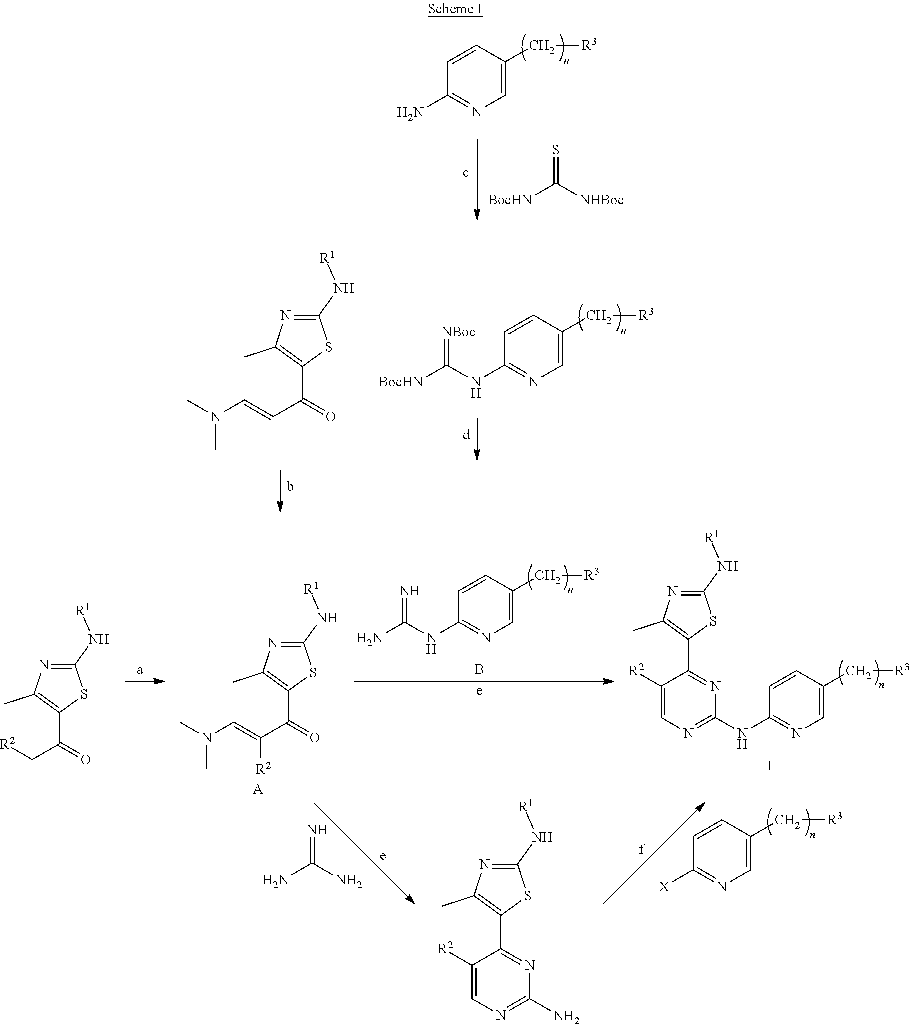

wherein the general reaction conditions are: (a) DMF-DMA or Bredereck's reagent, reflux; (b) Select Fluor, MeOH; (c) Et₃N, HgCl₂, DCM; (d) TFA/DCM (1:1), reflux; (e) A, B, NaOH, 2-methoxyethanol, microwave and (f) Pd₂dba₃, xantphose, t-BuONa, dioxane, microwave.

With regard to the description of the synthetic method of scheme 1 above, it will be understood by those skilled in the art that all proposed reaction conditions, including choice of solvent, reaction atmosphere, reaction temperature, duration of the experiment and workup procedures, can be readily selected. Moreover, it will be understood by those skilled in the art that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the examples hereinafter. Alternatively, necessary starting materials may be obtainable by analogous procedures to those illustrated which are within the ordinary skill of those skilled in the art. Further, it will be appreciated that during the synthesis of the compounds, or during the synthesis of certain starting materials, it may be desirable to protect certain substituent groups to prevent their undesired reaction. Those skilled in the art will readily recognise when such protection is required, and how such protecting groups may be put in place, and later removed. Examples of protecting groups are described in, for example, Protective Groups in Organic Synthesis by Theodora Green (publisher: John Wiley & Sons). Protecting groups may be removed by any convenient method well known to those skilled in the art as appropriate for the removal of the protecting group in question, such methods being chosen so as to effect removal of the protecting group with the minimum disturbance of groups elsewhere in the molecule. Thus, if reactants include, for example, groups such as amino, carboxyl or hydroxyl, it may be desirable to protect the group in some of the reactions mentioned herein.

In addition, those skilled in the art will be able to select appropriate reaction conditions to use in the coupling reaction of the compound of formula A or formula B shown in scheme 1. However, typically, the reaction will be carried out in anhydrous conditions and in the presence of an inert atmosphere, such as argon or nitrogen. The reaction may also be carried out an elevated temperature, such as, for example, within the range of 80 to 180° C. for a suitable time period of, for example, 20 minutes to 48 hours. Suitably, the reaction is carried out under microwave heating, for example, at 80 to 180° C. for 20 minutes to 1.5 hour.

The resultant compound can be isolated and purified using techniques well known to those skilled in the art.

The methods and uses of the disclosure are hereinafter further described with reference to the following, non-limiting examples and accompanying figures.

### EXAMPLES

Example 1 Synthesis of representative compounds

### General

¹H and ¹³C NMR spectra were recorded at 300 K on a Bruker AVANCE III 500 spectrometer (¹H at 500 MHz). ¹H NMR spectra were referenced to ¹H signals of residual non-deuterated solvents (or tetramethylsilane). High resolution mass spectra were recorded on an AB SCIEX TripleTOF® 5600 mass spectrometer, and ionisation of all samples was carried out using ESI. The purity of compounds was determined by analytical HPLC, and was greater than 95%. Analytic HPLC was carried out on a Shimadzu Prominence UFLC (UltraFast Liquid Chromatograph) system with a CBM-20A communications bus module, a DGU-20A_{5R} degassing unit, an LC-20AD liquid chromatograph pump, an SIL-20A_{III′} autosampler, an SPD-M20A photo diode array detector, a CTO-20A column oven and a Phenomenex Kinetex 5u C18 100 A 250 mm×4.60 mm column using Method A (gradient 5 to 95% MeOH containing 0.1% FA over 7 min, followed by 95% MeOH containing 0.1% FA over 13 min at a flow rate of 1 mL/min), Method B (gradient 5 to 95% MeCN containing 0.1% FA over 7 min followed by 95% MeCN containing 0.10% FA over 13 min, at a flow rate of 1 mL/min).

5-(2-((5-(4-(dimethylamino)piperidin-1-yl)pyridin-2-yl)amino)-5-fluoropyrimidin-4-yl)-N,4-dimethylthiazol-2-amine (1): To a solution of crude 1-(5-(4-(dimethylamino)piperidin-1-yl)pyridin-2-yl)guanidine trifluoroacetate (524 mg, 2.00 mmol) in 2-methoxyethanol (3 mL) were added ((E)-3-(dimethylamino)-2-fluoro-1-(4-methyl-2-(methylamino)thiazol-5-yl)prop-2-en-1-one (243 mg, 1.00 mmol) and NaOH (80.0 mg, 2.00 mmol). The reaction mixture was heated at 180° C. for 1h under microwave irradiation, cooled down to room temperature, and then concentrated under reduced pressure. The residue was purified by chromatography (silica gel, DCM ramping to DCM:MeOH 90:10 with constant addition of 0.5 ml of 32% ammonia) to give 1 as a brown solid (76 mg, 17.2%). ¹H NMR (DMSO-d₆) δ 1.50 (q, 2H, J 11.0), 1.84 (d, 3H, J 11.0), 2.21 (s, 7H), 2.47 (s, 3H, thiazole-CH₃), 2.64 (t, 2H, J 11.0), 2.86 (t, 3H, J 3.5), 3.63 (d, 1H, J 11.0), 7.39 (app d, 1H, J 7.0), 7.92 (d, 1H, J 9.0), 7.98 (s, 1H), 8.10 (1H, J 4.0), 8.41 (s, 1H), 9.43 (s, 1H). HRMS (ESI): m/z 443.2136 [M+H]⁺.

N-cyclopentyl-5-(2-((5-((4-ethylpiperazin-1-yl)methyl)pyridin-2-yl)amino)-5-fluoropyrimidin-4-yl)-4-methylthiazol-2-amine (2): To a solution of 5-(2-amino-5-fluoropyrimidin-4-yl)-N-cyclopentyl-4-methylthiazol-2-amine (200 mg, 0.68 mmol) in dioxane (3 mL) were added 1-((6-bromopyridin-3-yl)methyl)-4-ethylpiperazine (233. mg, 0.82 mmol), Pd₂dba₃ (31 mg, 0.034 mmol), xantphose (41 mg, 0.07 mmol) and t-BuONa (98 mg, 1.02 mmol) and heated under microwave irradiation at 150° C. for 1 h. The reaction mixture was cooled to room temperature and concentrated under reduced pressure. The residue was purified by chromatography (silica gel, DCM ramping to DCM:MeOH=93:7) to give 2 as an orange solid (100 mg, 29%). ¹H NMR (DMSO-d₆) δ 0.99 (t, 3H, J 7.0), 1.49-1.59 (m, 4H), 1.64-1.72 (m, 2H), 1.90-1.97 (m, 2H), 2.38 (s br, 10H), 2.48 (d, 3H, J 2.5), 3.42 (s, 2H), 3.95-3.98 (m, 1H), 7.64 (dd, 1H, J 8.5 & 2.0), 8.10 (d, 1H, J 8.5), 8.16 (d, 1H, J 2.0), 8.27 (d, 1H, J 7.0), 8.46 (d, 1H, J 3.5), 9.77 (s, 1H). HRMS (ESI): m/z 497.2601 [M+H]⁺.

N-Cyclopentyl-4-methyl-5-(2-((5-(piperazin-1-yl) pyridin-2-yl) amino) pyrimidin-4-yl) thiazol-2-amine (3): To a mixture of crude 1-(5-(piperazin-1-yl)pyridin-2-yl)guanidine trifluoroacetate (441 mg, 2.00 mmol) and (E)-1-(2-(cyclopentylamino)-4-methylthiazol-5-yl)-3-(dimethylamino) prop-2-en-1-one (279 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography (silica gel, DCM ramping to DCM:MeOH=92:8) and recrystallised with DCM and MeOH to give 3 as a dark yellow solid (70.0 mg, 16%). m.p. 210-213° C. ¹H NMR (DMSO-d₆) 1.49-1.68 (m, 7H), 1.89-1.94 (m, 2H), 2.46 (s, 3H), 2.85 (t, 4H, J 4.5), 3.02 (t, 4H, J 5.0), 3.98 (m, 1H), 6.90 (d, 1H, J 5.5), 7.36 (dd, 1H, J 9.0 & 3.0), 7.98 (d, 1H, J 3.0), 8.07 (d, 1H, J 9.0), 8.18 (d, 1H, J 7.0), 8.33 (d, 1H, J 5.5), 9.33 (s, 1H). IRMS (ESI): m:Z 437.2222 [M+H]⁺.

N-Cyclopentyl-5-(2-((5-(4-ethylpiperazin-1-yl)pyridin-2-yl)amino)pyrimidin-4-yl)-4-methylthiazol-2-amine (4): To a mixture of crude 1-(5-(4-ethylpiperazin-1-yl)pyridin-2-yl)guanidine trifluoroacetate (496 mg, 2.00 mmol) and (E)-1-(2-(cyclopentylamino)-4-methylthiazol-5-yl)-3-(dimethylamino) prop-2-en-1-one (279 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography (silica gel, DCM ramping to DCM:MeOH 96:4) and recrystallised from MeOH to give 4 as a yellow solid (117 mg, 25%). ¹H NMR (CDCl₃) δ 1.14 (t, 3H, J 7.0), 1.56-1.76 (m, 6H), 2.06-2.12 (m, 2H), 2.49 (q, 2H, J 7.5), 2.54 (s, 3H), 2.64 (s, 3H), 3.19 (t, 4H, J 4.5), 3.14 (t, 4H, J 5.0), 3.86 (app s, 1H), 5.77 (s, 1H), 6.84 (d, 1H, J 5.0), 7.34 (dd, 1H, J 9.0 & 3.0), 7.94 (d, 1H, J 3.0), 7.94 (s, 1H), 8.01 (d, 1H, J 3.0), 8.26 (d, 1H, J 9.0), 8.33 (d, 1H, J 5.5). HRMS (ESI): m/z 465.2541 [M+H]⁺.

2-((5-(4-acetylpiperazin-1-yl)pyridin-2-yl)amino)-4-(4-methyl-2-(methylamino)thiazol-5-yl)pyrimidine-5-carbonitrile (5): To a solution of crude 1-(5-(4-acetylpiperazin-1-yl)pyridin-2-yl)guanidine trifluoroacetate (315 mg, 1.20 mmol) in 2-methoxyethanol (4 mL) were added tert-butyl (E)-(5-(2-cyano-3-(dimethylamino)acryloyl)-4-methylthiazol-2-yl)(methyl)carbamate (350 mg, 1.00 mmol) and NaOH (82.0 mg, 2.40 mmol). The reaction mixture was heated at 180° C. for 90 min under microwave irradiation, cooled down to room temperature, and then concentrated under reduced pressure. The residue was purified by chromatography (silica gel, DCM ramping to DCM:MeOH=90:10 with consecutive addition of 32% aqueous ammonia, up to 3%). The solid was washed with DCM and MeOH, then filtered to give 5 as a pale yellow solid (157 mg, 35%). H NMR (DMSO-d₆) δ 2.04 (s, 3H), 2.40 (s, 3H), 2.87 (s, 3H), 3.10 (t, 2H, J 5.0), 3.16 (t, 2H, J 5.0), 3.58 (t, 4H, J 5.0), 7.46 (dd, 1H, J 9.5 & 3.0), 7.90 (d, 1H, J 9.0), 8.06 (d, 1H, J 3.0), 8.26 (q, 1H, J 3.0), 8.75 (s, 1H), 10.33 (s, 1H). HRMS (ESI): m/z 450.1844 [M+H]⁺.

N-cyclopentyl-5-(2-((5-((4-ethylpiperazin-1-yl)methyl)pyridin-2-yl)amino)pyrimidin-4-yl)-4-methylthiazol-2-amine (6): To a solution of 5-(2-aminopyrimidin-4-yl)-N-cyclopentyl-4-methylthiazol-2-amine (275 mg, 1.00 mmol) in dioxane (3 mL) were added 1-((6-bromopyridin-3-yl)methyl)-4-ethylpiperazine (341 mg, 1.2 mmol), Pd₂dba₃ (45.8 mg, 0.05 mmol), xantphose (58 mg, 0.1 mmol) and t-BuONa (144 mg, 1.5 mmol) and heated under microwave irradiation at 150° C. for 1 h. The reaction mixture was cooled to room temperature and concentrated under reduced pressure. The residue was purified by chromatography (silica gel, DCM ramping to DCM:MeOH:NH₄OH=9:1:0.3) and recrystallised with DCM and MeOH to give 6 as a white solid (200 mg, 42%). ¹H NMR (CDCl₃) δ 1.09 (t, 3H, J 7.0), 1.58-1.76 (m, 6H), 2.08-2.14 (m, 2H), 2.43 (q, 2H, J 7.0, CH₂CH₃), 2.55 (s br, 11H), 3.48 (s, 2H), 3.86-3.92 (m, 1H), 5.42 (d, 2H, J 7.0), 6.90 (d, 1H, J 5.5), 7.68 (dd, 1H, J 9.0 & 2.5), 7.89 (s, 1H), 8.19 (d, 1H, J 2.0), 8.35-8.38 (m, 2H). HRMS (ESI): m/z 479.2703[M+H]⁺.

5-(2-((5-(4-aminopiperidin-1-yl)pyridin-2-yl)amino)-5-fluoropyrimidin-4-yl)-N,4-dimethylthiazol-2-amine (7): Compound 7 was obtained by reacting 1-(5-(4-aminopiperidin-1-yl)pyridin-2-yl)guanidine trifluoroacetate (702 mg, 3.00 mmol) and (469 mg, 2.00 mmol) and ((E)-3-(dimethylamino)-2-fluoro-1-(4-methyl-2-(methylamino)thiazol-5-yl)prop-2-en-1-one (243 mg, 1.00 mmol) as an orange solid (40.0 mg, 10%). ¹H NMR (DMSO-d₆) δ 1.75-1.80 (m, 2H), 2.45 (d, 3H, J 2.0), 2.62 (t, 2H, J 6.0), 2.85 (t, 2H, J 5.5), 3.45 (t, 2H, J 5.0), 3.53 (t, 2H, J 6.0), 7.13 (dd, 1H, J 9.0 & 3.0), 7.78 (s, 1H), 7.79 (d, 1H, J 4.5), 8.08 (q, 1H, J 4.5), 8.37 (d, 1H, J 3.5), 9.21 (s, 1H). HRMS (ESI): m/z 415.1821 [M+H]⁺.

5-(2-((5-(4-aminopiperidin-1-yl)pyridin-2-yl)amino)pyrimidin-4-yl)-N-cyclopentyl-4-methylthiazol-2-amine (8): To a mixture of 1-(5-(4-aminopiperidin-1-yl)pyridin-2-yl)guanidine trifluoroacetate (702 mg, 3.00 mmol) and (E)-1-(2-(cyclopentylamino)-4-methylthiazol-5-yl)-3-(dimethylamino) prop-2-en-1-one (558 mg, 2.00 mmol) in 2-methoxy ethanol (5 mL) was added NaOH (160.0 mg, 4.00 mmol). The reaction mixture was heated at 180° C. under microwave irradiation for 2 h, cooled to room temperature and concentrated under reduced pressure. The residue was purified by chromatography to give 8 as a yellow solid (90 mg, 10%). ¹H NMR (CDCl₃) δ 1.50-1.77 (m, 10H), 1.95 (d, 2H, J 10.5), 2.07-2.13 (m, 2H), 2.54 (s, 3H), 2.75-2.85 (m, 3H), 3.53-3.56 (m, 2H), 3.85-3.91 (m, 1H), 5.43 (d, J 5.0, 1H), 6.84 (d, 1H, J 5.5), 7.34 (dd, 1H, J 9.0 & 3.0), 7.75 (s, 1H), 8.00 (d, 1H, J 3.0), 8.25 (d, 1H, J 9.0), 8.32 (d, 1H, J 5.5). HRMS (ESI): m/z 451.2415 [M+H]⁺.

N-cyclopentyl-5-(2-((5-morpholinopyridin-2-yl)amino)pyrimidin-4-yl)-4-(trifluoromethyl)thiazol-2-amine (9): To a mixture of 1-(5-morpholinopyridin-2-yl)guanidine trifluoroacetate (442 mg, 2.00 mmol) and (E)-1-(2-(cyclopentylamino)-4-methylthiazol-5-yl)-3-(dimethylamino)-2-fluoroprop-2-en-1-one (297 mg, 1.00 mmol) in 2-methoxy ethanol (3 mL) was added NaOH (80.0 mg, 2.00 mmol). The reaction mixture was heated at 180° C. under microwave irradiation for 1 h, cooled to room temperature and concentrated under reduced pressure. The residue was purified by chromatography to give 9 as a brown solid (120 mg, 26%). ¹H NMR (DMSO-d₆) δ 1.50-1.57 (m, 4H), 1.66-1.69 (m, 2H), 1.90-1.95 (m, 2H), 2.47 (d, 1H, J 12.5), 3.09 (t, 4H, J 5.0), 3.75 (t, 4H, 15.0), 3.96 (m, 1H), 7.42 (dd, 1H, J 9.0 & 3.0), 7.96 (d, 1H, J 9.0), 7.98 (d, 1H, J 3.0), 8.24 (d, 1H, J 7.0), 8.41 (d, 1H, J 7.0), 9.52 (s, 1H). HRMS (ESI): m 456.1976 [M+H].

### Example 2 Biological Activity

### Kinase Assays

Eurofins Pharma Discovery or Reaction Biology Corporation Kinase Profiler services were used to measure inhibition of CDKs and other kinases by radiometric assay (RIA). Inhibition of CDK4/Cyclin D1, CDK6/Cyclin D3 and CDK9/T1 were also determined in-house using ADP Glo Kinase assays (Promega Corporation, Madison Wis., United States of America). Briefly, the kinase reaction for CDK4/Cyclin D1 and CDK6/Cyclin D3 was performed with kinase reaction buffer (40 nM Tris base pH 7.5, 20 mM MgCl₂, 0.4 mM DTT), 0.1 mg/ml BSA and RB-CTF substrate (retinoblastoma protein1 C-terminal fraction). For CDK9/CyclinT1, the kinase reaction was performed with standard assay buffer and Kinase Dilution Buffer and RBER-IRStide substrate. Serial dilutions of 1:3 were prepared for test compounds for 10 concentrations (from 10 μM to 0.5 nM). The kinase reactions were started by addition of ATP, incubated for 40 min at 37° C. and then stopped by adding 10 μL of ADP Glo reagent. After incubation at room temperature (RT) in the dark for 40 min, 20 μL of kinase detection reagent was added per well and incubated for 40 min. Luminescence was measured using an EnVision Multilabel plate reader (PerkinElmer, Buckinghamshire, United Kingdom). Positive and negative controls were performed in the presence and absence of CDK kinases, respectively. Half-maximal inhibition (IC₅₀) values were calculated using a 4-parameter logistic non-linear regression model with Graphpad prism (Version 6.0). Apparent inhibition constants (Kᵢ) values were calculated from Kₘ (ATP) and IC₅₀ values for the respective kinases. The results of representative compounds are shown in Table 2.

TABLE 2
Inhibition of cyclin-dependant kinases

| | CDK inhibition Kᵢ (μM) or % remaining enzymatic activity at 10 μM | | | | | |
|---|---|---|---|---|---|---|
| Compound | CDK1/B | CDK2/A | CDK4/D1 | CDK6/D3 | CDK7/H | CDK9/T1 |
| 1 | >1 | >1 | 0.002 | 0.279 | >1 | >1 |
| 2 | >1 | 0.620 | 0.0002 | 0.003 | 0.630 | >1 |
| 3 | >1 | >1 | 0.001 | 0.034 | >1 | 0.220 |
| 4 | >1 | 0.355 | 0.002 | 0.011 | 0.780 | 0.141 |
| 5 | >1 | >1 | 0.004 | 0.030 | >1 | >1 |
| 6 | >1 | 0.416 | 0.006 | 0.009 | 0.630 | >1 |
| 7 | >1 | 0.206 | 0.003 | 0.032 | >1 | >1 |
| 8 | >1 | 0.278 | 0.001 | 0.008 | 0.282 | 0.508 |
| 9 | >1 | 0.104 | 0.006 | 0.02 | >1 | >1 |

### Cell Culture

All three GBM cell lines used in this example, namely U87, U251 and T98G, were cultured in Eagle's Minimum Essential Medium (EMEM) with 10% fetal bovine serum in 75 cm² flasks at 37° C. and 5% CO₂. All cell lines were confirmed to be mycoplasma-free prior to any experiment setup.

### MTT Proliferation Assays and Combination Studies

Representative compounds from Example 1, were subjected to standard MTT (3-(4,5-dimethylthiazol-2-yl)-2,5-diphenyltetrazolium bromide) and resazurin assays on solid tumour cell lines (including GBM cell lines) and leukemia cell lines, respectively, as previously reported (Wang S et al., J Med Chem 47:1662-1675, 2004; and Diab S. et al. CheMedChem 9:962-972, 2014). Compound concentrations required to inhibit 50% of cell growth (GI₅₀) were determined using GraphPad Prism 8 (GraphPad Software, Inc.; San Diego, Calif., United States of America). The results are shown in Table 3.

TABLE 3

| | Antiproliferative activity (72 h, GI₅₀ μM) | | | | | | | | | | |
|---|---|---|---|---|---|---|---|---|---|---|---|
| | Leukaemia | Breast | Colorectal | Glioblastoma | | Lung | | Ovarian | Pancreas | Prostate | Melanoma |
| Compd | MV4-11 | MB453 | Colo205 | T98G | U87 | U251 | H460 | A2780 | PANC1 | LNCap | M229 |
| 1 | 0.11 | 0.33 | 2.32 | 2.96 | 4.46 | 5.52 | 3.28 | 0.07 | 1.32 | 0.62 | — |
| 2 | 0.01 | 0.62 | 1.55 | 4.18 | 2.17 | 5.09 | 0.41 | 0.04 | 1.21 | 0.28 | 0.83 |
| 3 | 0.02 | 0.07 | — | — | — | — | — | — | — | — | — |
| 4 | 0.01 | 0.29 | — | — | — | — | — | — | — | — | — |
| 5 | 0.17 | 0.81 | — | — | — | — | — | — | — | — | — |
| 6 | 0.02 | 0.07 | 4.38 | — | — | — | — | — | — | — | — |
| 7 | 0.08 | 0.36 | — | — | — | — | — | — | — | — | — |
| 8 | 0.03 | 0.10 | — | — | — | — | — | — | — | — | — |
| 9 | 0.29 | 1.45 | — | — | — | — | — | — | — | — | — |

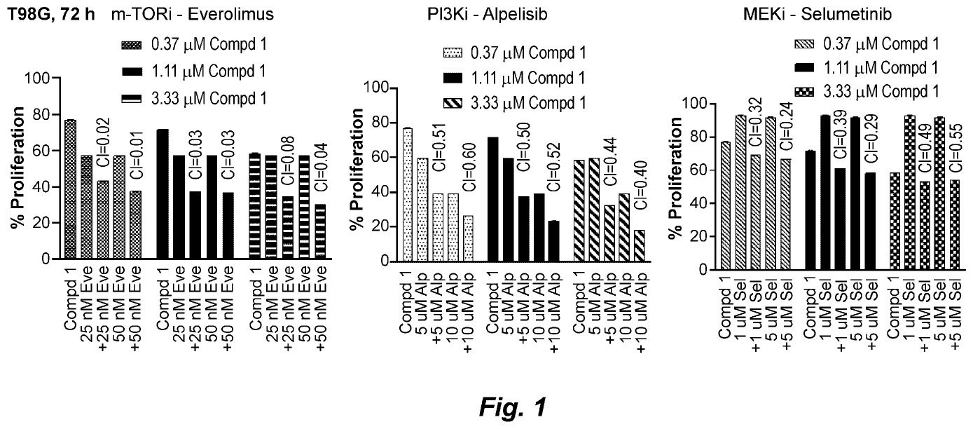

For combination studies, cells were treated with 10 μL everolimus (an inhibitor of the mTOR), alpelisib (an inhibitor of PI3K) or selumetinib (an inhibitor of the MEK) and 10 μL of one of the CDK4/6 inhibitors of the present disclosure with various concentrations for 72 h. Meanwhile, 50 μL MTT was added to each well, and the absorbance was measured at 550 nm by EnVision® multilabel plate reader (Buckinghamshire, UK) following a 3.5 h incubation. The outcomes were determined by the Chou-Talalay method using CompuSyn v 1.0 (ComboSyn, NJ, United States of America). Values of combination index (CI)<1, 1 and >1 were considered as synergistic, additive and antagonistic drug interactions, respectively (Chou T C et al., Trends Pharmacol Sci 4:450-454, 1983). The results are provided at FIG. 1. The combination treatments provided a greater level of inhibitory activity on proliferation relative to the single agent treatments. There was evidence of a synergistic level of inhibition as showing by CI<1.

### Apoptosis Assays

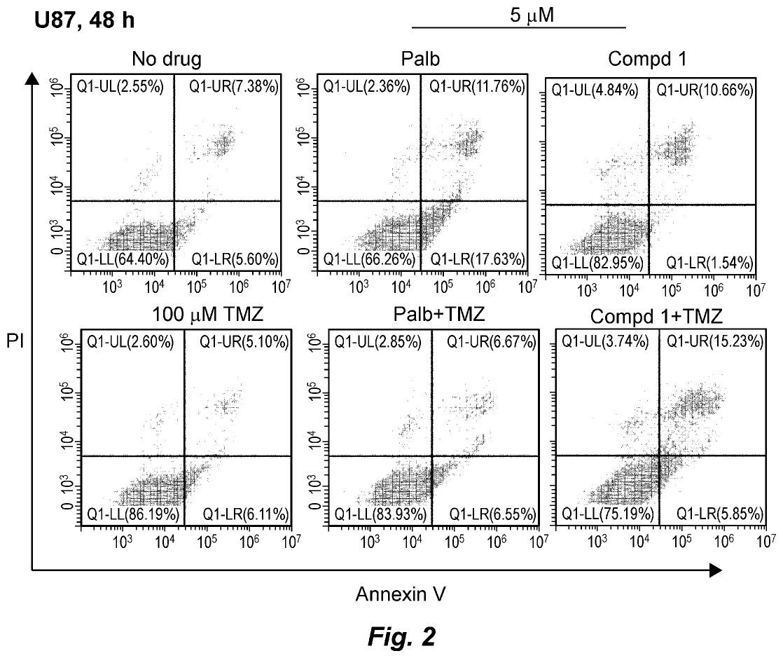

The ability of compound 1 to induce apoptosis in GBM cells was investigated using the annexin V-FITC apoptosis detection kit I (BD Pharmingen Inc.; San Diego, Calif., United States of America) according to the manufacturer's protocol. Cell pellets were washed twice with 1 mL cold PBS and centrifuged at 300×g for 5 mins. Following the removal of supernatants, cell pellets were stained with 100 μL 1× annexin V binding buffer, 3 μL annexin V-FITC and 3 μL propidium iodide staining solution, and incubated in the dark at room temperature for 15 mins. The percentage of apoptic cells was measured by a CytoFLEX flow cytometer. GBM U87 cells were treated with 5 μM palbociclib (“Palb”) or compound 1 (with and without 5 μM temozolomide (TMZ)), and thereafter the cells were assayed, after 48 hours. The results in FIG. 2 showed that compound 1 (with or without TMZ) is capable of inducing apoptosis in GBM cells to a greater level than the control anti-cancer agent, palbociclib (with or without TMZ).

### Western Blot Analysis

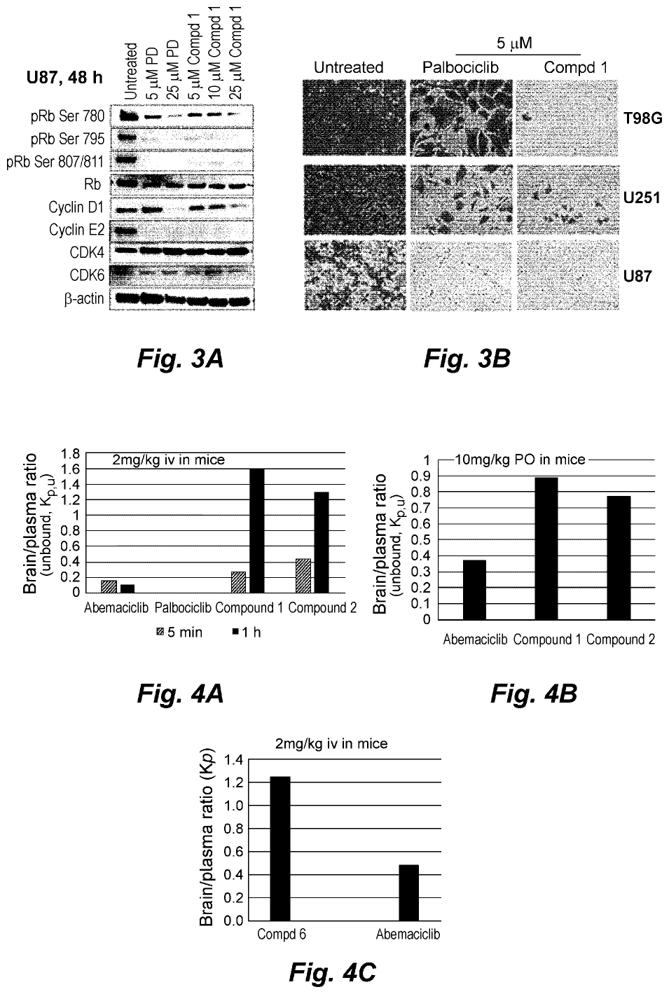

Cells were seeded at a density of 3×10⁵ with 10 mL fresh medium on a sterile culture dish and incubated at 37° C. 5% CO₂ for overnight prior to compound treatment. After 24 h, cells were harvested with PBS, centrifuged at 300×g for 5 mins, and lysed with 100 μL lysis buffer (25 mM 4-(2-hydroxyethyl)-1-piperazineethanesulphonic acid, 300 mM sodium chloride, 1.5 mM magnesium chloride, 0.5% sodium deoxycholate, 20 mM p-glycerophosphate, 1% Triton X-100, 0.1% sodium dodecyl sulphate, 0.2 mM EDTA, 0.5 mM dithiothreitol, 1 mM sodium orthovanadate and 25× protease inhibitor cocktail). Protein lysate was collected after 10 mins of maximum speed centrifugation at 4° C. The protein concentration of cell lysates was determined through performing a BSA standard curve using the Bio-Rad DC protein assay kit II (Bio-Rad, Sydney, NSW, Australia) according to the manufacturer's protocol. 30 μg protein was measured, mixed with 3× loading buffer and denatured at 95° C. for 5 mins using T100 thermal cycler (Bio-Rad). Molecular mass of each sample was then separated by gel electrophoresis on a 4-20% polyacrylamide gel at 120V for 1 h. The resolved proteins were transferred onto nitrocellulose membranes and blocked with 5% skimmed milk in Tris-buffered saline and Tween 20 (TBST) for 1 h at room temperature. Antibodies were diluted in 5% skimmed milk in TBST. Membranes were incubated with primary antibody at −20° C. for overnight, washed four times with TBST, treated with secondary antibody for an hour at room temperature and washed four times with TBST again. Chemiluminescence on the membrane was then developed using Amersham ECL prime/select western blotting detection reagent (GE Healthcare, Sydney, NSW, Australia). Band intensity was determined using a ChemiDoc™ multiplexing imaging system (Bio-Rad). Antibodies used were obtained from Cell Signalling Technology (Danvers, Mass., United States of America): p-Rb (S780) #9307, p-Rb (S795) #9301, p-Rb (S807/811) #8516, Rb #9309, cyclin D1 #2978, cyclin E #4132, CDK4 #12790, CDK6 #13331, β-actin #4970, HRP-linked anti-mouse IgG #7076 and HRP-linked anti-rabbit IgG #7074. FIG. 3A shows that compound 1 (and palbociclib, a positive control) reduces the level of phosphorylated Rb proteins (pRb Ser780, pRbSer795, pRb807/811), confirming its cellular CDK4/6 inhibition.

### In Vitro Tumorigenicity Assays

A density of 200 to 400 cells/well was seeded in a twelve-well plate and incubated for overnight prior to compound treatment. Each well was changed with fresh medium containing compound 1 (or positive control palbociclib) every 2-3 days. After 10 days, cells were washed with PBS, fixed and stained with crystal violet staining solution (0.05% crystal violet, 1% formaldehyde, 1% PBS, 1% ethanol); the results are shown in FIG. 3B. Compound 1 inhibits the growth and proliferation of all three GBM cell lines.

### Brain Uptake Assays

The propensity of the compounds from Example 1 to be uptaken by the brain (thereby showing that they are capable of crossing the BBB) was investigated using the method as described (T J Raub et al., supra). Briefly, Balb/C mice were administered with compound 1 or compound 2 intravenously. At 5 min and 1 h post-dose, cardiac blood was collected, and cerebral hemispheres excised. For the oral dosing experiments, mice were given drug and blood plus cerebral hemispheres were collected at 0.5, 1, 2, 4, 6, and 24 h post-dose. Concentrations of drug in homogenised brains and plasma were measured by LC-MS/MS; those in brain homogenate were converted to brain concentrations (expressed per g of brain tissue) and corrected for an average measured plasma volume of 16 μL/g brain tissue. Unbound fraction in plasma and brain was determined using Centrifree® Ultrafiltration Devices. Value of K_{p,u} for each drug was compared against a set guidelines for neurotherapeutic agents (Kulkarni A D et al. Expert Opin Drug Deliv 13:85-92, 2016). Control experiments were conducted using equivalent doses of palbociclib and/or abemaciclib. The results, shown in FIG. 4, are expressed as K_{p,u}: brain to plasma ratio of the unbound drug concentration, which is usually categorised into the following bands: K_{p,u}<0.1, unable to cross the BBB; K_{p,u} 0.3-0.5 sufficient access from the BBB; K_{p,u}>1.0 freely cross the BBB (Kulkarni et al., supra). Compounds 1, 2 and 6 demonstrated a significantly greater propensity to cross the BBB than the control anti-cancer agents.

### In Vivo Anti-Tumour Efficacy

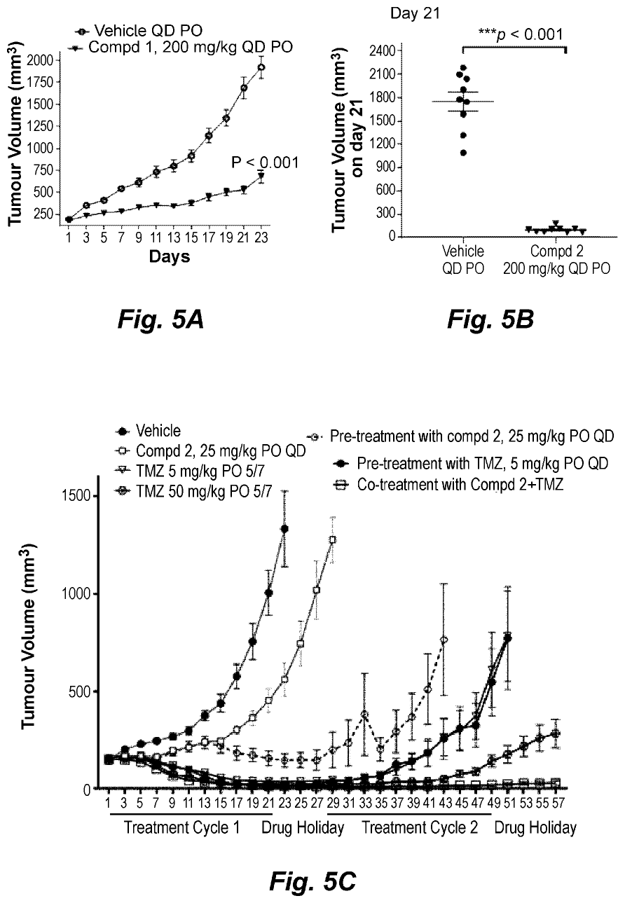

Compound 1 and compound 2 were also investigated for in vivo antitumour activity in subcutaneous xenograft models. Briefly, U87 GBM cells were harvested and resuspended in a 1:1 mixture of serum-free media and matrigel, and 5×10⁶ cells injected subcutaneously into the rear flank of 5-6-week-old CD1 nu/nu female mice. When the mean tumour volume reached 150-200 mm³, animals were randomised to treatment groups (n=10 per group) and treated orally with vehicle (0.5% carboxymethyl cellulose in water), compound 1 or compound 2 daily for 21 days with the doses shown in FIGS. 5A and 5B. Mice were observed daily for signs of toxicity and weight loss, and tumour volume measured every other day. Both compounds markedly reduced tumour growth compared to vehicle treatment (T/C=310% and 6% on day 21 for compound 1 and compound 2, respectively; p<0.001 as determined by two-tailed t-test) without any overt toxicity.

In another study, U87 xenografted mice (n=8 per group) were treated with: (1) vehicle, (2) compound 2 (25 mg/kg, PO, daily), (3 and 4) TMZ (5 mg/kg or 50 mg/kg, PO, 5 day/week), (5) pre-treatment with compound 2 (25 mg/kg, PO, daily ×10), followed by TMZ (5 mg/kg, PO, 5 day/week ×2), (6) pre-treatment with TMZ (5 mg/kg, PO, 5 day/week ×2) followed by compound 2 (25 mg/kg, PO, daily ×10), and (7) co-treatment with compound 2 and TMZ, respectively. All treatment groups were completed by day 21, which were followed by a 7-day drug holiday before the second cycle of treatment started from day 29 (FIG. 5B). Compound 2 demonstrated significant anti-tumour efficacy both as a single agent and in combination with TMZ (p<0.001) without any overt toxicity when compared to vehicle-treated group.

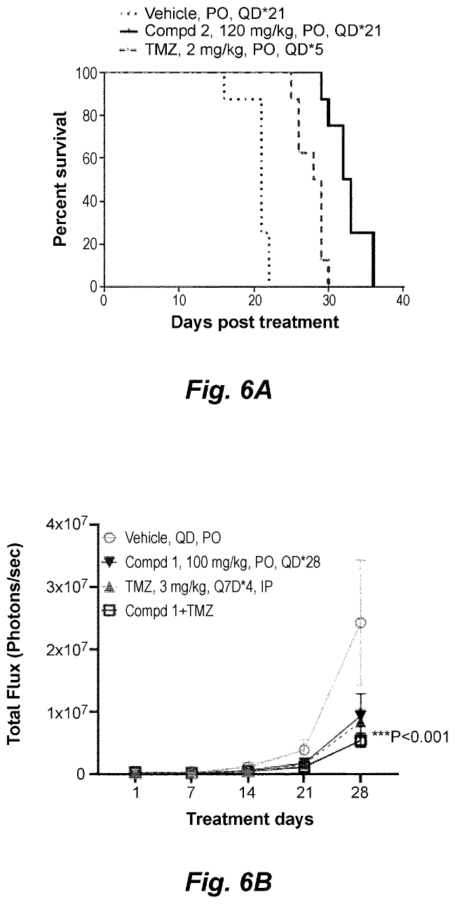

In vivo anti-tumour efficacy of compound 1 and compound 2 was also investigated in GBM orthotopic mouse xenograft models. For example, compound 2 was administered orally once daily for 21 days to NSG mice (n=8) (The Jackson Laboratory, Ben Harbor, Me., United States of America) orthotopically engrafted with the U87 GBM cells per each treatment cohort. TMZ was given orally once daily for 5 days and was used as a positive control. The disease burden was measured by a non-invasive bioluminescent whole-body imaging technique. The treatment with compound 2 resulted in a significant inhibition in tumour growth (TGI=81.4%, p<0.001) on day 21 as compared to vehicle-treated mice. An increase in life span ratio [ILS=(DaysT−DaysC)/DaysC, where DaysC=days survived by control group and DaysT=days survived by treatment group] of 154.8% for compound 2 treated mice was observed when compared to vehicle control (FIG. 6A).

In a separate study, NSG mice (n=8) orthotopically engrafted with GBM patient-derived G4T cells were administered with vehicle, compound 1, TMZ, and co-treatment of compound 1 and TMZ, respectively, as shown in FIG. 6B. The disease burden of each of the treatment groups was measured by bioluminescent whole-body imaging. Compound 1 demonstrated significant inhibition of tumour growth either as a single agent or in combination with TMZ (p<0.001, FIG. 6B).

### CONCLUSION

Compounds of formula I were shown to inhibit CDK4/6 and possess anti-proliferative activity against GBM cell lines in both in vitro and in vivo settings. Further, the representative compounds, namely compound 1 (5-(2-((5-(4-(dimethylamino)piperidin-1-yl)pyridin-2-yl)amino)-5-fluoropyrimidin-4-yl)-N,4-dimethylthiazol-2-amine), compound 2 (N-cyclopentyl-5-(2-((5-((4-ethylpiperazin-1-yl)methyl)pyridin-2-yl)amino)-5-fluoropyrimidin-4-yl)-4-methylthiazol-2-amine) and compound 6 (N-cyclopentyl-5-(2-((5-((4-ethylpiperazin-1-yl)methyl)pyridin-2-yl)amino)pyrimidin-4-yl)-4-methylthiazol-2-amine) were found to be capable of crossing the blood brain barrier and highly efficacious in the GBM tumour-bearing animal models, indicating that the compounds of formula I have excellent prospects to provide an effective therapeutic agent for the treatment of proliferative cell diseases and conditions of the CNS such as GBM.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the present disclosure is not restricted in its use to the particular application described. Neither is the present disclosure restricted in its preferred embodiment with regard to the particular elements and/or features described or depicted herein. It will be also appreciated that the disclosure is not limited to the embodiment or embodiments disclosed, but is capable of numerous rearrangements, modifications and substitutions without departing from the scope of the disclosure as set forth and defined by the following claims.

Please note that the following claims are provisional claims only, and are provided as examples of possible claims and are not intended to limit the scope of what may be claimed in any future patent applications based on the present application. Integers may be added to or omitted from the example claims at a later date so as to further define or re-define the various aspects of the present disclosure.

1. A method of treating a proliferative cell disease or condition of the central nervous system (CNS) in a subject, comprising administering to the subject a therapeutically effective amount of a compound of formula I shown below: 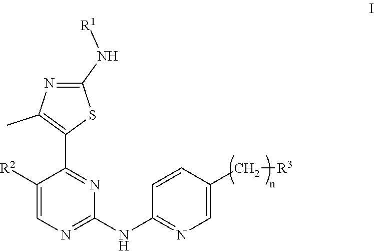
wherein:
R¹ is selected from H, alkyl, aryl, aralkyl, alicyclic, heterocyclic, halogen, NO₂, CN, CF₃, OH, O-alkyl, O-aryl, COOH, CO-alkyl, CO-aryl, CONH₂, CONH-alkyl, CONH-aryl, and CONH-alicyclic;
R² is selected from H, alkyl, halogen, NO₂, CN, CF₃, OH, O-alkyl, and NH₂;
R³ is selected from heterocyclic including at least one N heteroatom, NH-alkyl, NH-aryl, N-(alkyl)₂, N-(aryl)₂, and N-(alkyl)(aryl); and
n is an integer selected from the range of 0 to 3; and
wherein said alkyl, aryl, aralkyl, alicyclic and heterocyclic groups may be optionally substituted with one or more groups selected from alkyl, halogen, CN, OH, O-methyl, NH₂, NH-alkyl, N(alkyl)₂, COOH, COH, CO(alkyl), CONH₂ and CF₃;
or a pharmaceutically acceptable salt, solvate or prodrug thereof, optionally in combination with a pharmaceutically acceptable carrier, diluent and/or excipient.

2. The method of claim 1, wherein R¹ is H, alkyl or a C₃₋₆ cycloalkyl or a heterocyclic group comprising one or two N, O or S heteroatoms.

3. The method of claim 2, wherein R¹ is H, methyl, ethyl, cyclopropyl or cyclopentyl.

4. The method of claim 1, wherein R² is H, alkyl, CN or halogen.

5. The method of claim 4, wherein R² is H, methyl, ethyl, CN or F.

6. The method of claim 1, wherein R³ is a heterocyclic group, optionally substituted with one or more groups selected from alkyl, NH₂, NH-alkyl, N(alkyl)₂, COH and CO(C₁₋₃ alkyl).

7. The method of claim 6, wherein R³ is selected from the following:

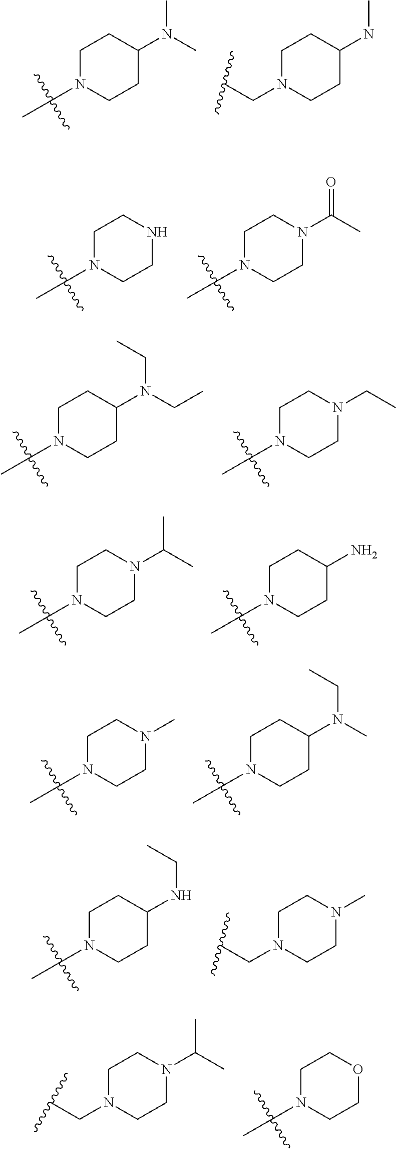

8. The method of claim 14, wherein n is 0, 1 or 2.

9. The method of claim 1, wherein the compound is:
5-(2-((5-(4-(dimethylamino)piperidin-1-yl)pyridin-2-yl)amino)-5-fluoropyrimidin-4-yl)-N,4-dimethylthiazol-2-amine;
N-cyclopentyl-5-(2-((5-((4-ethylpiperazin-1-yl)methyl)pyridin-2-yl)amino)-5-fluoropyrimidin-4-yl)-4-methylthiazol-2-amine;
N-cyclopentyl-4-methyl-5-(2-((5-(piperazin-1-yl)pyridin-2-yl)amino)pyrimidin-4-yl)thiazol-2-amine;
N-cyclopentyl-5-(2-((5-(4-ethylpiperazin-1-yl)pyridin-2-yl)amino)pyrimidin-4-yl)-4-methylthiazol-2-amine;
2-((5-(4-acetylpiperazin-1-yl)pyridin-2-yl)amino)-4-(4-methyl-2-(methylamino)thiazol-5-yl)pyrimidine-5-carbonitrile;
N-cyclopentyl-5-(2-((5-((4-ethylpiperazin-1-yl)methyl)pyridin-2-yl)amino)pyrimidin-4-yl)-4-methylthiazol-2-amine;
5-(2-((5-(4-aminopiperidin-1-yl)pyridin-2-yl)amino)-5-fluoropyrimidin-4-yl)-N,4-dimethylthiazol-2-amine;
5-(2-((5-(4-aminopiperidin-1-yl)pyridin-2-yl)amino)pyrimidin-4-yl)-N-cyclopentyl-4-methylthiazol-2-amine
N-cyclopentyl-5-(5-fluoro-2-((5-morpholinopyridin-2-yl)amino)pyrimidin-4-yl)-4-methylthiazol-2-amine
5-(2-((5-(4-(ethylamino)piperidin-1-yl)pyridin-2-yl)amino)-5-fluoropyrimidin-4-yl)-N,4-dimethylthiazol-2-amine;
5-(2-((5-(4-(ethyl(methyl)amino)piperidin-1-yl)pyridin-2-yl)amino)-5-fluoropyrimidin-4-yl)-N,4-dimethylthiazol-2-amine;
5-(5-fluoro-2-((5-((4-methylpiperazin-1-yl)methyl)pyridin-2-yl)amino)pyrimidin-4-yl)-N,4-dimethylthiazol-2-amine;
5-(5-fluoro-2-((5-((4-isopropylpiperazin-1-yl)methyl)pyridin-2-yl)amino)pyrimidin-4-yl)-N,4-dimethylthiazol-2-amine; or
5-(2-((5-(4-(diethylamino)piperidin-1-yl)pyridin-2-yl)amino)-5-fluoropyrimidin-4-yl)-N,4-dimethylthiazol-2-amine.

10. The method of claim 1, wherein the compound, or a pharmaceutically acceptable salt, solvate or prodrug thereof, is formulated for intravenous and/or oral administration.

11. The method of claim 1, wherein the compound, or a pharmaceutically acceptable salt, solvate or prodrug thereof, is administered to the subject in combination with temozolamide (TMZ) or other kinase inhibitor.

12. The method of claim 11, wherein the other kinase inhibitor is selected from inhibitors of PI3K, mTOR and/or MEK.

13. The method of claim 1, wherein the proliferative cell disease or condition is glioblastoma (GBM), medulloblastoma, primary central nervous system (CNS) lymphoma, astrocytoma, ependymona, oligodendroglioma, or metastatic brain tumour.

14. (canceled)

15. (canceled)

16. The method of claim 2, wherein R¹ is a C₁₋₆ alkyl or a saturated or unsaturated 5- or 6-membered cyclic group comprising one or two N, O or S heteroatoms.

17. The method of claim 4, wherein R² is C₁₋₆ alkyl or F.

18. The method of claim 6, wherein R³ is a saturated or unsaturated 5- or 6-membered group comprising one or two N heteroatoms, optionally substituted by NH₂, NH-methyl, NH-ethyl, N(CH₃)₂, N(CH₂CH₃), N(CH₃)(CH₂CH₃)), or COCH₃.
